(19) United States
(12) Patent Application Publication
(10) Pub. No.: US 2024/0148967 A1
(43) Pub. Date: May 9, 2024

(54) TREATMENT OF PAIN ASSOCIATED WITH TOTAL KNEE ARTHROPLASTY WITH SUSTAINED-RELEASE LIPOSOMAL ANESTHETIC COMPOSITIONS

(71) Applicant: Pacira Pharmaceuticals, Inc., Parsippany, NJ (US)

(72) Inventors: Roy Winston, Parsippany, NJ (US); Mary DiGiorgi, Parsippany, NJ (US)

(21) Appl. No.: 18/104,979

(22) Filed: Feb. 2, 2023

Related U.S. Application Data
(60) Provisional application No. 63/422,146, filed on Nov. 3, 2022.

(51) Int. Cl. A61M 5/158; A61K 9/00; A61K 9/127; A61K 31/445; A61M 5/42; A61P 23/02

(52) U.S. Cl. CPC A61M 5/158; A61K 9/0085; A61K 9/127; A61K 31/445; A61M 5/427; A61P 23/02

(57) ABSTRACT

In some embodiments provided herein are methods of administering an adductor canal block in a patient, wherein the methods include: (a) selecting an entry point of an injection needle in a leg of the patient; (b) inserting the injection needle into the leg of the patient at the entry point; (c) identifying a first nerve in the leg of the patient; (d) administering to the first nerve saline and a pharmaceutical composition; (e) identifying a second nerve in the leg of the patient; (f) administering to the second nerve saline and the pharmaceutical composition; wherein the first nerve and second nerve are selected from the group consisting of the nerve to vastus medialis (NVM) and the saphenous nerve, and wherein the first nerve is not the second nerve, and wherein the pharmaceutical composition comprises multivesicular liposomes.

### CROSS-REFERENCE TO RELATED APPLICATIONS

This application claims the benefit of U.S. Provisional Application No. 63/422,146, filed Nov. 3, 2022, which is incorporated by reference herein in its entirety.

### BACKGROUND

Postsurgical pain is one of the most common forms of acute pain, which is the normal physiological response to tissue insult or injury and has adaptive value by serving as a warning of danger or damage. Most acute pain is either treatable or avoidable, especially when it occurs in a clinical setting. However, if acute pain is poorly or inappropriately treated, it may progress to chronic pain. Thus, effective postsurgical pain control is a critical element in patient recovery following surgery, as the majority of patients may experience significant pain, particularly in the first few days. Improved postsurgical pain management contributes to better healing, faster patient mobilization, shortened hospital stays, and reduced healthcare costs.

Knee surgery, including knee arthroplasty, is usually used to manage pain and disability in the knee. However, total knee arthroplasty (TKA) is recognized as a painful orthopedic procedure, wherein over half of those undergoing TKA experience severe postoperative pain. Opioids are commonly used to manage post-operative orthopedic pain. Opioid use carries a risk of developing tolerance and dependence. Thus, an important goal following surgery is to improve analgesia while reducing opioid consumption.

Multimodal pain management approaches are recommended by professional societies to improve analgesia, reduce opioid use, and decrease opioid-related adverse events (AEs) following knee surgery. It is recommended that protocols include long-acting neuraxial opioids together with scheduled acetaminophen and nonsteroidal anti-inflammatory drugs (NSAIDs). However, most many orthopedic pain patients still request opioids after cesarean delivery for breakthrough pain. Thus, there continues to be a need for methods of treating pain associated with surgery, including knee surgery in a subject.

### SUMMARY

Provided herein are methods of administering to the adductor canal of a patient a pharmaceutical composition for post-operative analgesia, comprising: (a) selecting an entry point of an injection needle in a leg of a patient by ultrasound transducer; (b) inserting the injection needle into the patient at the entry point; (c) identifying a first nerve in the leg of the patient; (d) administering to the first nerve saline and a pharmaceutical composition; (e) identifying a second nerve in the leg of the patient; (f) administering to the second nerve saline and the pharmaceutical composition; wherein the first nerve and second nerve are selected from the group consisting of the nerve to vastus medialis (NVM) and the saphenous nerve, and wherein the first nerve is not the second nerve, and wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome, thereby administering to the adductor canal of the patient the pharmaceutical composition for post-operative analgesia.

Provided herein are methods of administering to the adductor canal of a human patient a pharmaceutical composition for post-operative analgesia, comprising: (a) selecting an entry point of an injection needle in a leg in a patient by ultrasound transducer; (b) advancing a needle tip of the injection needle within a region between a vastus medialis muscle and a sartorius muscle of the patient along a trajectory that extends between the entry point and a superficial femoral artery; (c) identifying a first nerve in the leg of the patient; (d) administering to the first nerve through the injection needle saline and about 10 mL of a pharmaceutical composition; (e) identifying a second nerve in the leg of the patient; (f) administering to the second nerve through the injection needle saline and about 10 mL of the pharmaceutical composition; wherein the first nerve and second nerve are selected from the group consisting of the nerve to vastus medialis (NVM) and the saphenous nerve, and wherein the first nerve is not the second nerve, and wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome, thereby administering to the adductor canal of the human patient the pharmaceutical composition for post-operative analgesia.

Provided herein are methods of treating post-operative knee pain in a patient, comprising: (a) selecting an entry point of an injection needle in a leg of the patient; (b) inserting the injection needle into the leg of the patient at the entry point; (c) identifying a first nerve in the leg of the patient; (d) administering to the first nerve saline and a pharmaceutical composition; (e) identifying a second nerve in the leg of the patient; (f) administering to the second nerve saline and the pharmaceutical composition; wherein the first nerve and second nerve are selected from the group consisting of the nerve to vastus medialis (NVM) and the saphenous nerve, and wherein the first nerve is not the second nerve, and wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome, thereby treating post-operative knee pain in the patient.

Provided herein are methods of administering an adductor canal block to a patient, comprising: (a) selecting an entry point of an injection needle in a leg of a patient by ultrasound transducer, wherein the entry point comprises a region adjacent to a superficial femoral artery in the middle of a sartorius muscle; (b) advancing a needle tip of the injection needle within a region between a vastus medialis muscle and a sartorius muscle of the patient along a trajectory that extends between the entry point and a superficial femoral artery; (c) identifying a nerve to vastus medialis (NVM) in the patient; (d) administering to the NVM through the injection needle saline and about 10 mL of a multivesicular liposome pharmaceutical composition; (e) identifying a saphenous nerve, wherein identifying a saphenous nerve in the patient comprises penetrating a vasto-adductor membrane with the needle tip and advancing the needle tip to a location anterior to the superficial femoral artery; (f) administering to the saphenous nerve through the injection needle saline and about 10 mL of a multivesicular liposome pharmaceutical composition to the saphenous nerve; wherein the multivesicular liposome pharmaceutical composition comprises: bupivacaine or a salt thereof; phosphoric acid; a lipid component comprising at least one amphipathic lipid and at least one neutral lipid lacking a hydrophilic head group; and, optionally, a cholesterol and/or a plant sterol, thereby administering the adductor canal block to the patient.

Further, in some embodiments the injection needle is connected to a peripheral nerve stimulator (PNS). In some embodiments, the PNS is tuned to 2 hertz and between 0.5 to 1.0 mA. In some embodiments, the PNS is used to identify the first nerve and/or the second nerve. In some embodiments, the PNS is used to identify the first nerve and the second nerve. In some embodiments, the first nerve is the nerve to vastus medialis (NVM) and the second nerve is the saphenous nerve. In some embodiments, identifying the entry point of the injection needle comprises the point at which a superficial femoral artery is in the middle of a sartorius muscle. In some embodiments, the injection needle is a 100 mm, 21-gauge needle. In some embodiments, the injection needle is insulated. In some embodiments, the injection needle into the leg of the patient comprises advancing a needle tip of the injection needle within a region between a vastus medialis muscle and a sartorius muscle of the patient along a trajectory that extends between the entry point and a superficial femoral artery. In some embodiments, the insertion of the injection needle into the leg of the patient does not comprise penetrating a vasto-adductor membrane. In some embodiments, insertion of the injection needle into the leg of the patient comprises penetrating a vasto-adductor membrane with the needle tip and advancing the needle tip to a location anterior to the superficial femoral artery. In some embodiments, the saline injection comprises no more than 1 to 2 mL of saline. In some embodiments, a syringe used for the saline injection is different from a syringe used for the pharmaceutical administration. In some embodiments, administering the pharmaceutical composition comprises administering about 10 mL of the pharmaceutical composition to each of the first nerve and the second nerve. In some embodiments, the method comprises administering about 20 mL total of the pharmaceutical composition. In some embodiments, the multivesicular liposomes comprise: bupivacaine or a salt thereof; phosphoric acid; a lipid component comprising at least one amphipathic lipid and at least one neutral lipid lacking a hydrophilic head group; and, optionally, a cholesterol and/or a plant sterol, wherein said multivesicular liposomes are made by a process comprising: (a) preparing a first aqueous component comprising phosphoric acid; (b) preparing a lipid component comprising at least one organic solvent, at least one amphipathic lipid, and at least one neutral lipid lacking a hydrophilic head group; (c) mixing said first aqueous component and said lipid component to form a water-in-oil emulsion, wherein at least one component comprises bupivacaine or a salt thereof; (d) mixing said water-in-oil emulsion with a second aqueous component to form solvent spherules; and (e) removing the organic solvent from the solvent spherules to form multivesicular liposomes encapsulating bupivacaine phosphate. In some embodiments, the methods further comprise administering local anesthetic in the space between the popliteal artery and the posterior capsule of the knee. In some embodiments, the local anesthetic comprises bupivacaine HCl. In some embodiments, the local anesthetic comprises 15 mL of 0.25% bupivacaine HCl. In some embodiments, administration of the local anesthetic uses a 100 mm insulated needle. In some embodiments, the space between the popliteal artery and the posterior capsule of the knee is determined by ultrasound. In some embodiments, administration of the local anesthetic includes administration anterior to the popliteal artery.

### BRIEF DESCRIPTION OF THE DRAWINGS

FIG. 1 shows a schematic of an exemplary injection location for a femoral nerve block.

FIG. 2 shows a schematic of an exemplary injection location for an adductor canal block.

FIG. 3 shows Table 1, a summary of the demographics of the participants of clinical trial #1.

FIG. 4 shows Table 2, a summary of the baseline characteristics of the participants in clinical trial #1.

FIG. 5 shows the time and events schedule of study procedures (screening through POD 14).

FIG. 6 shows Table 3, a summary of the area under the curve of numeric rating scale (NRS) pain intensity score at 0-96 hours post-surgery for participants in clinical trial #1.

FIG. 7 shows Table 4, a summary of the area under the curve of numeric rating scale (NRS) pain intensity score by time interval for participants in clinical trial #1.

FIG. 8 shows Table 5, a summary of total opioid consumption by time interval for participants in clinical trial #1.

FIG. 9 shows Table 6, a summary of time to first opioid consumption post-surgery for participants in clinical trial #1.

FIG. 10 shows Table 7, a summary of the worst and average numeric rating scale pain intensity scores over the last 24 hours from post-surgery day 1 through day 14 for participants in clinical trial #1.

FIG. 11 shows pain intensity between 0 and 96 hours, between EXPAREL admixed with bupivacaine hydrochloride (HCl; round circles) and bupivacaine HCl alone (plus signs) for participants in clinical trial #1. Patients receiving EXPAREL report less pain starting at approximately 18 hours after surgery.

FIG. 12 shows mean bupivacaine concentration over time. Subjects receiving EXPAREL admixed with bupivacaine hydrochloride exhibit an early and a late Cₘₐₓ, whereas a single peak in PK for those receiving bupivacaine hydrochloride alone for participants in clinical trial #1.

FIG. 13 shows Table 8, a summary of pharmacokinetic (PK) parameters for EXPAREL-admix patients and bupivacaine hydrochloride alone patients for participants in clinical trial #1.

FIG. 14 shows Table 9, a summary of time to onset and duration of motor and sensory block for EXPAREL-admix patients and bupivacaine hydrochloride alone patients for participants in clinical trial #1.

FIG. 15 shows Table 10, an overview of treatment-emergent adverse events between EXPAREL-admix patients and bupivacaine hydrochloride alone patients for participants in clinical trial #1.

FIG. 16 shows Table 11, a summary of treatment-emergent adverse events occurring in ≥5% of subjects in any cohort for participants in clinical trial #1.

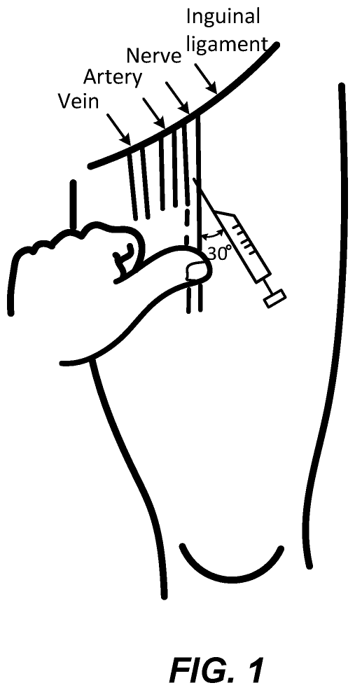

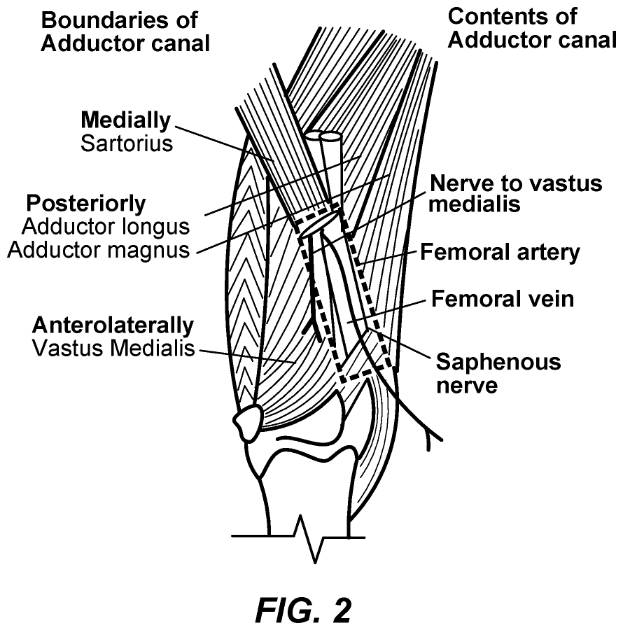

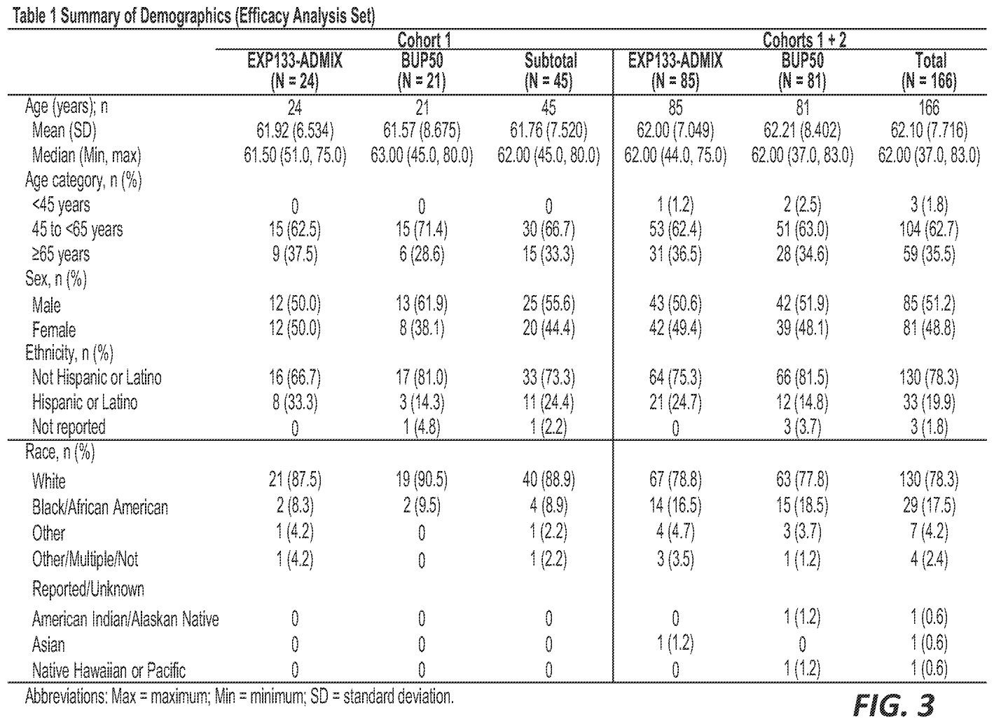

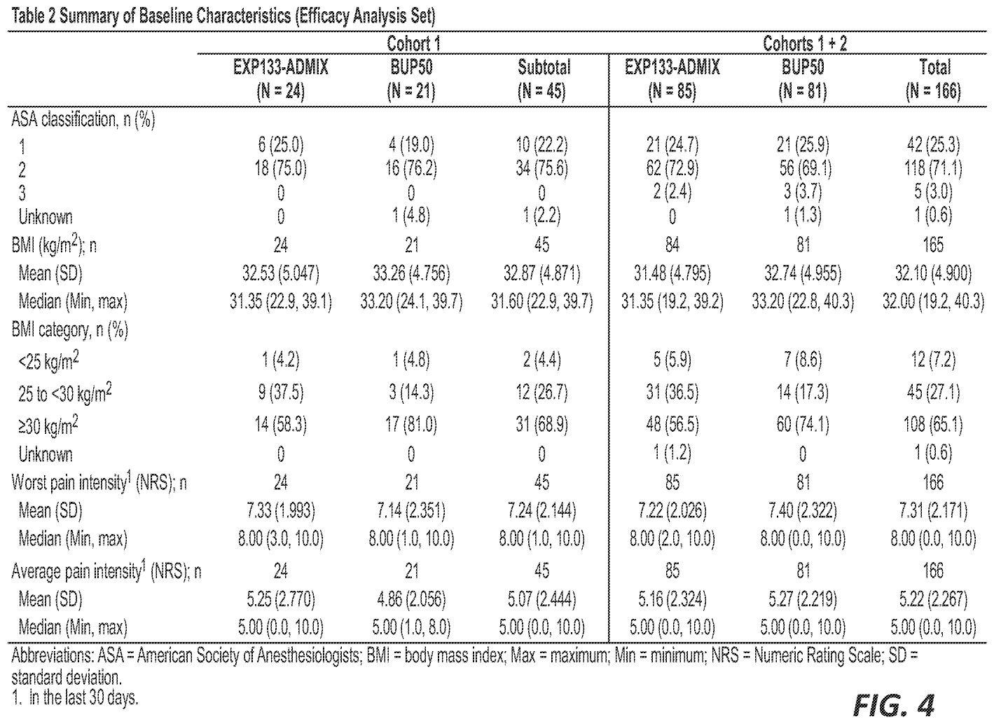

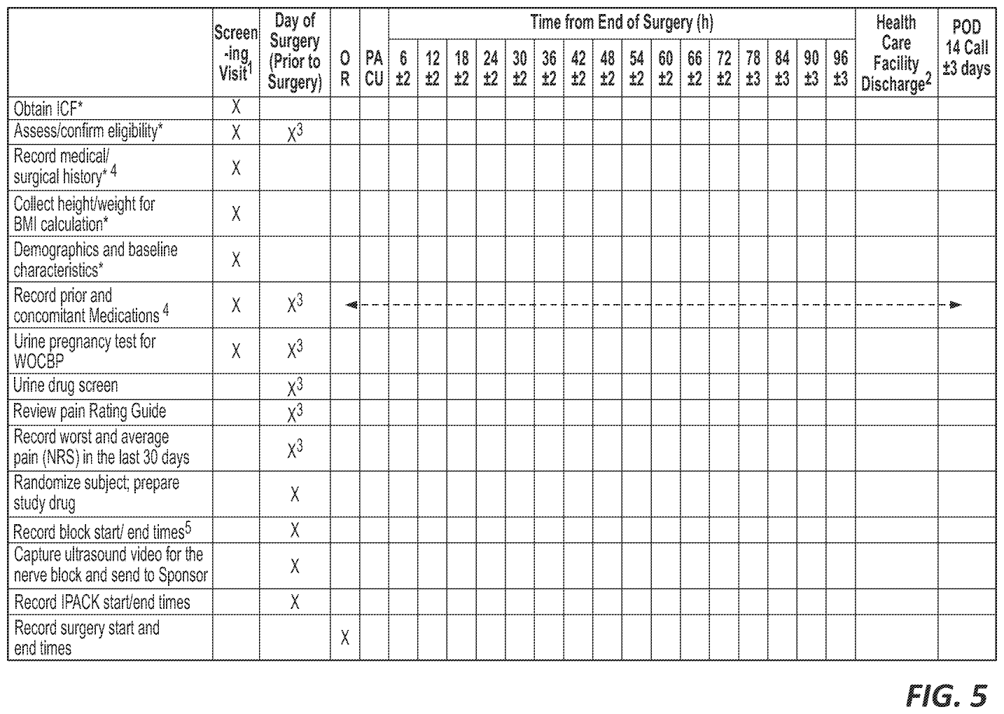

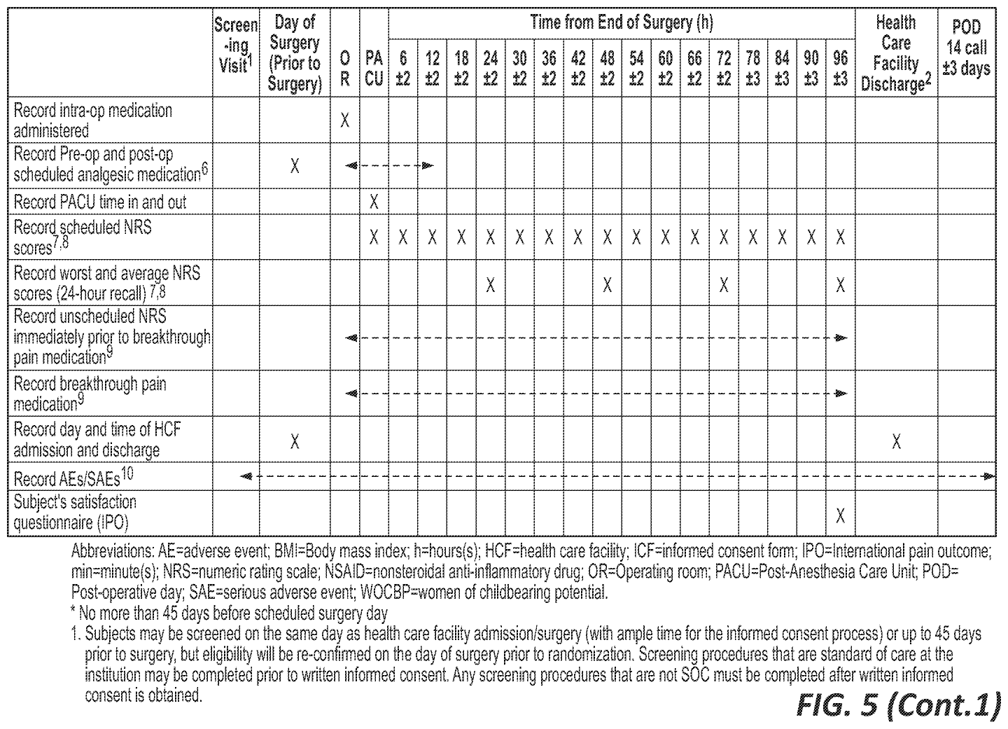

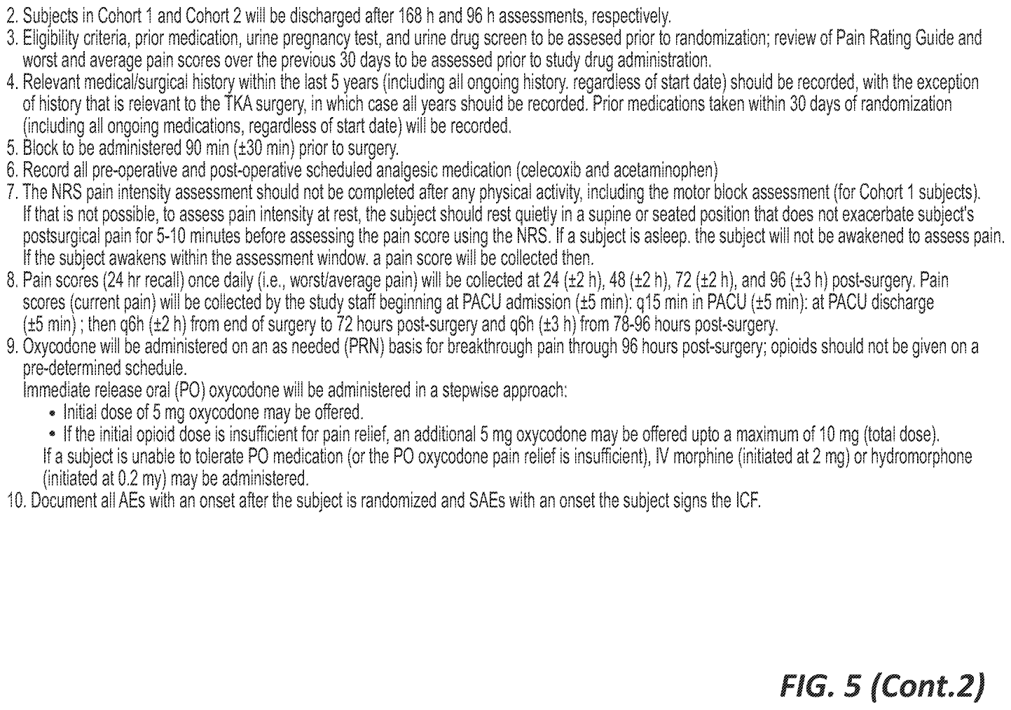

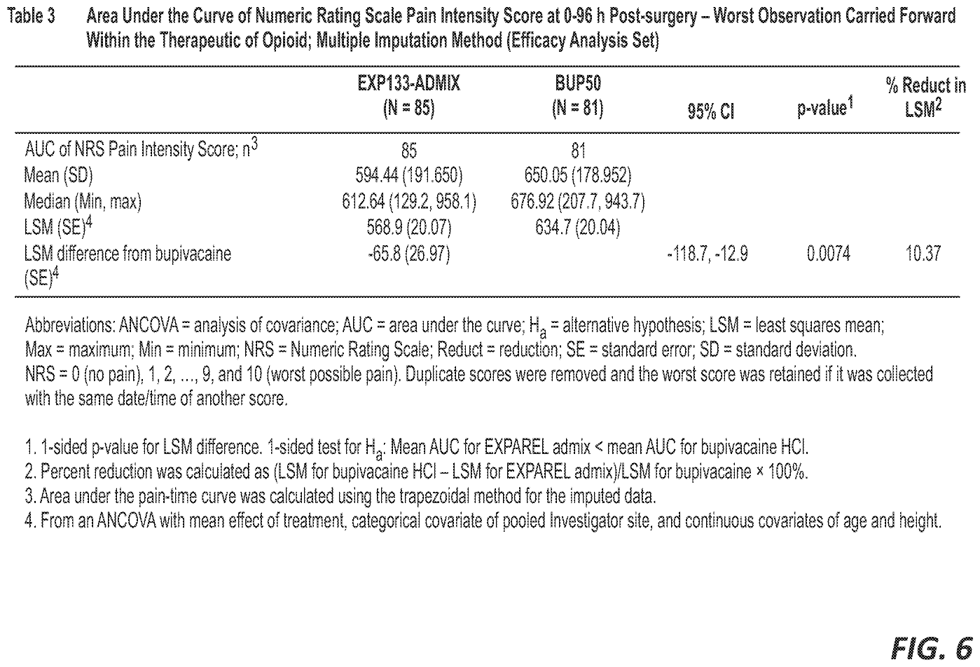

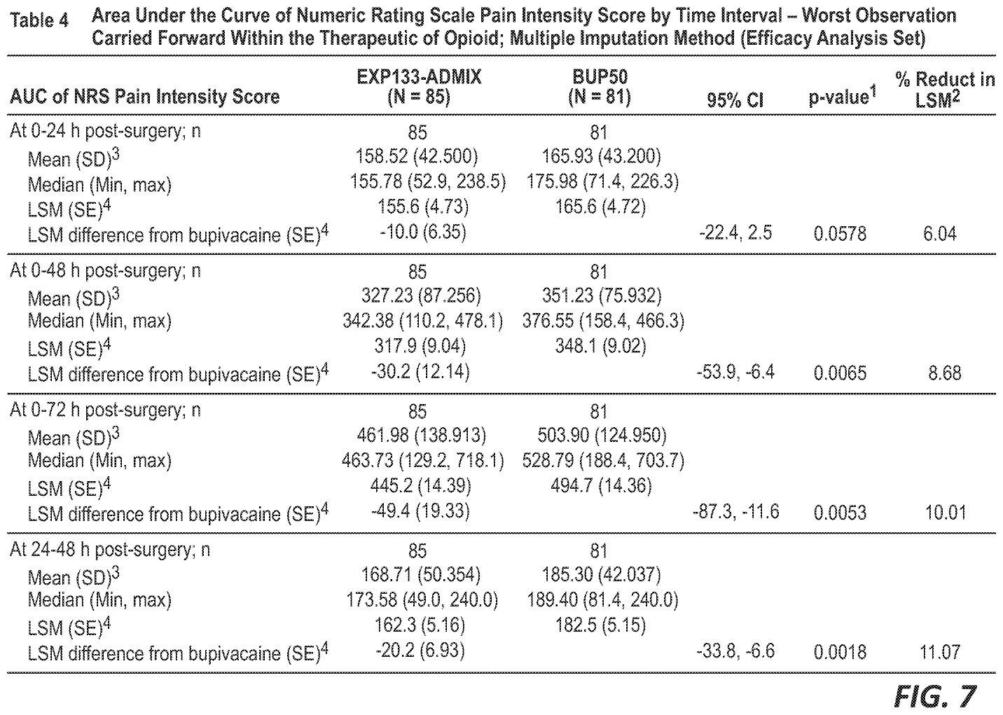

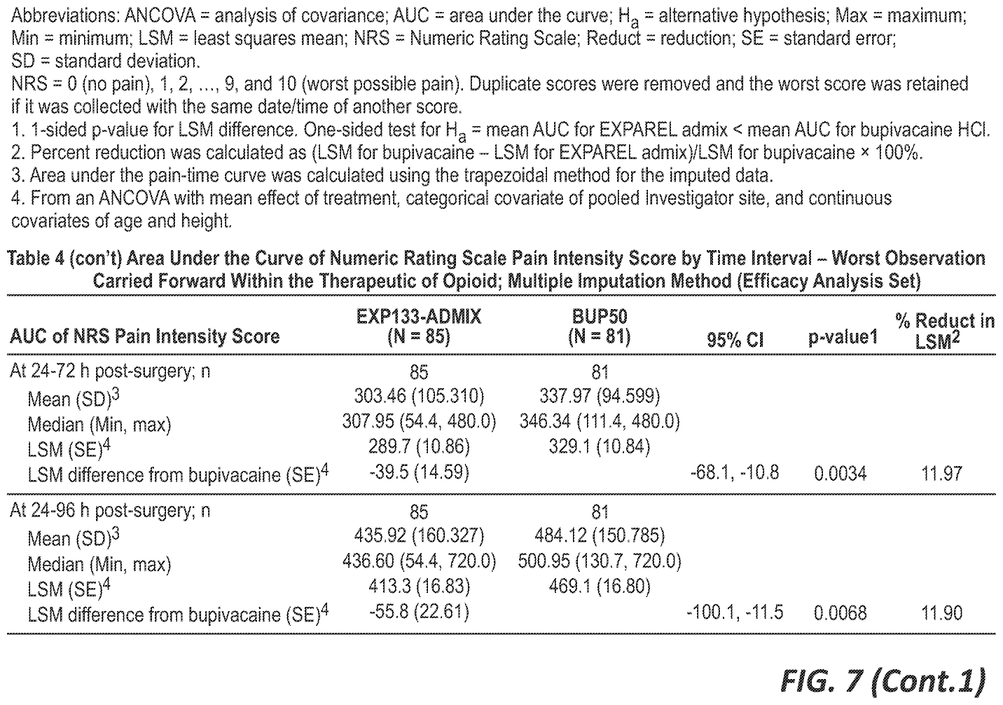

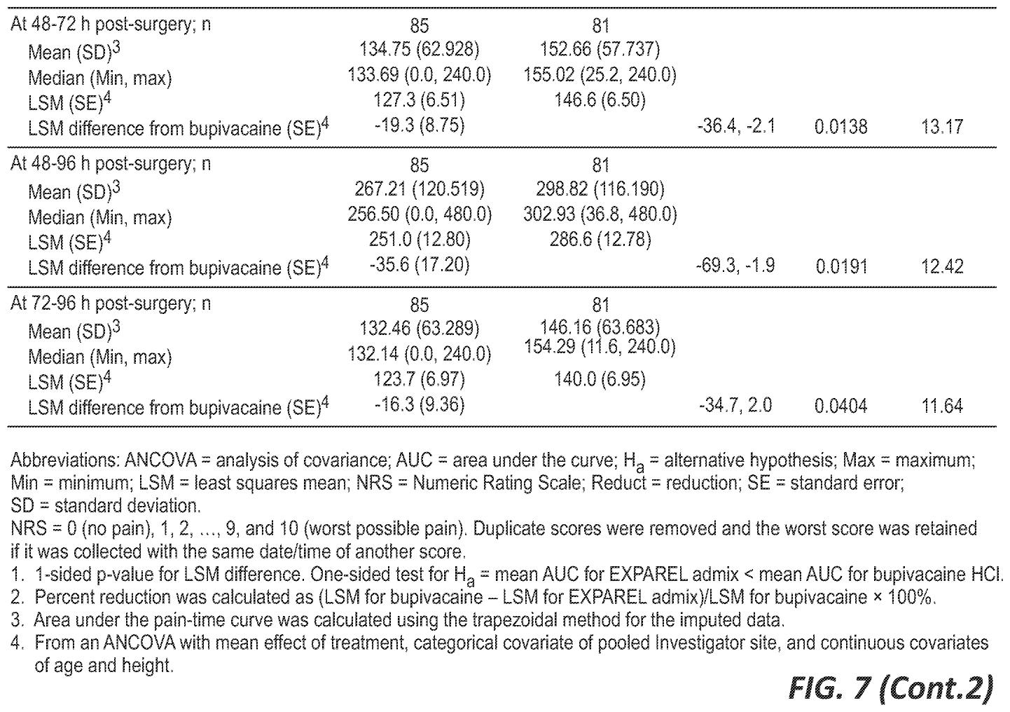

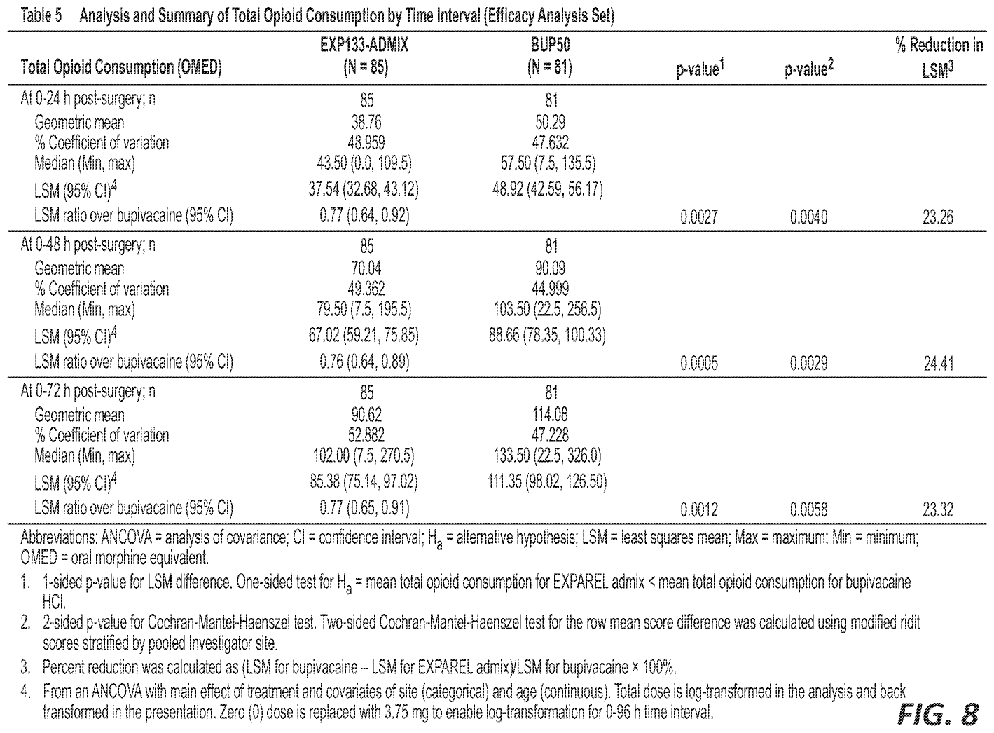

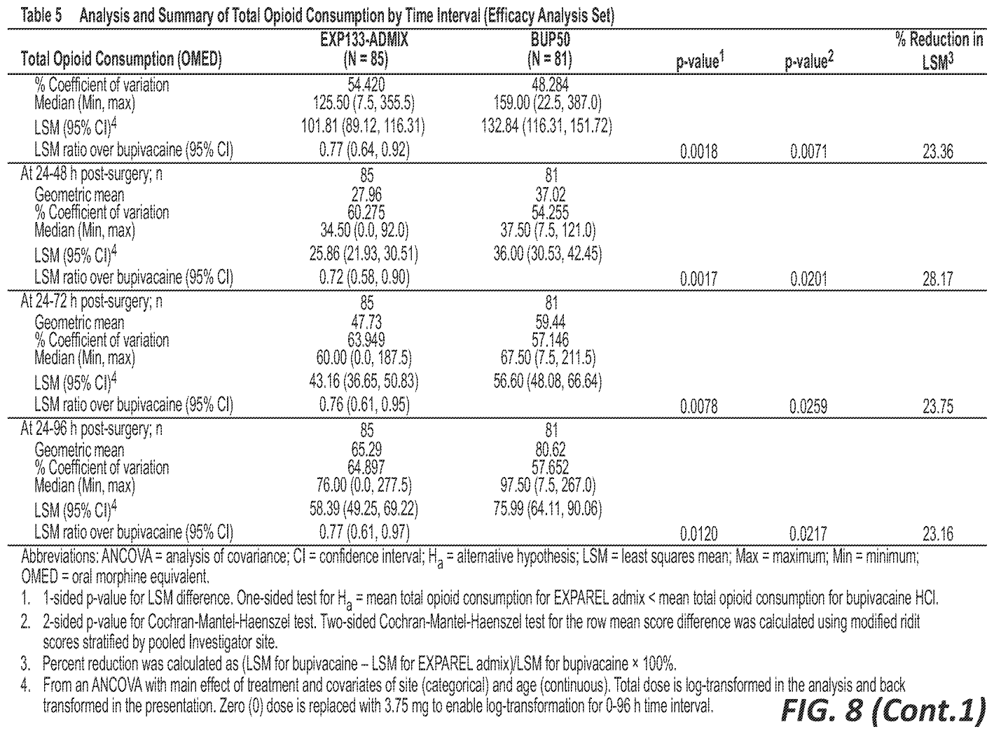

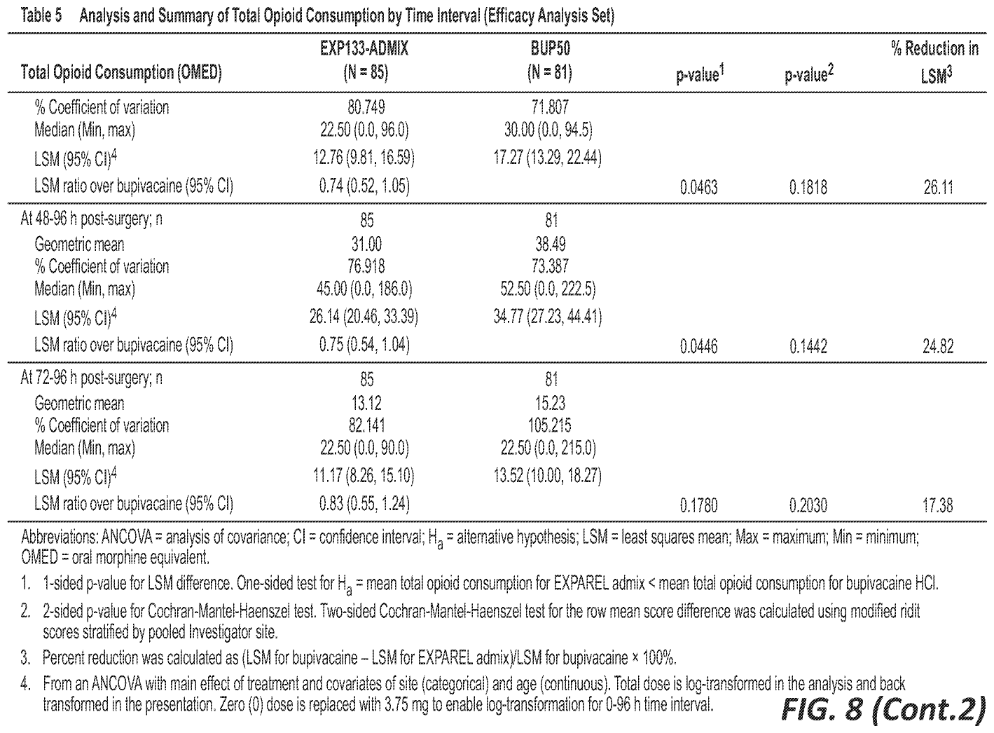

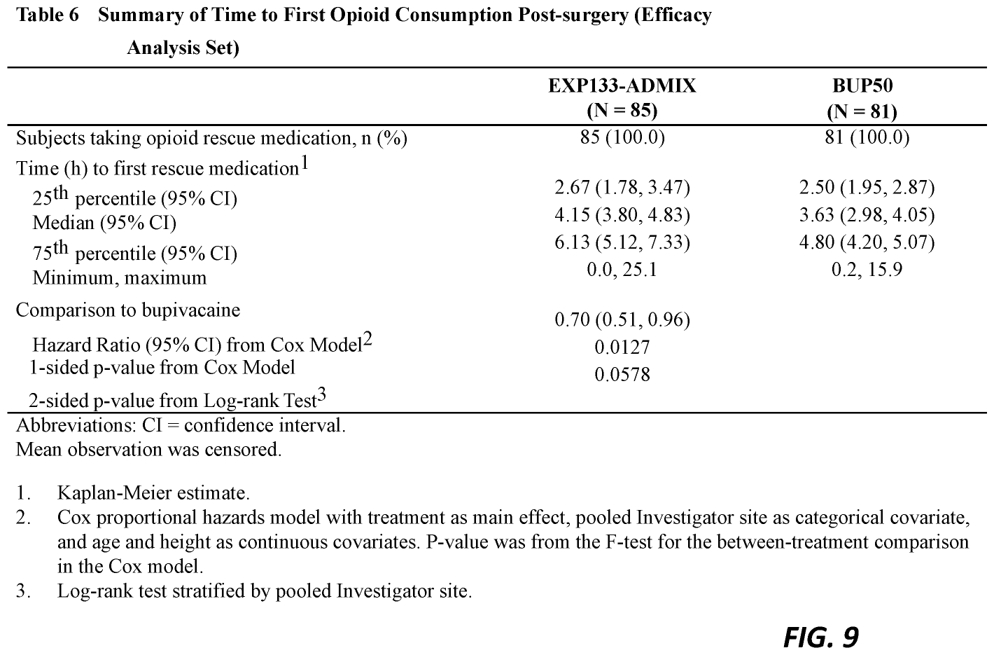

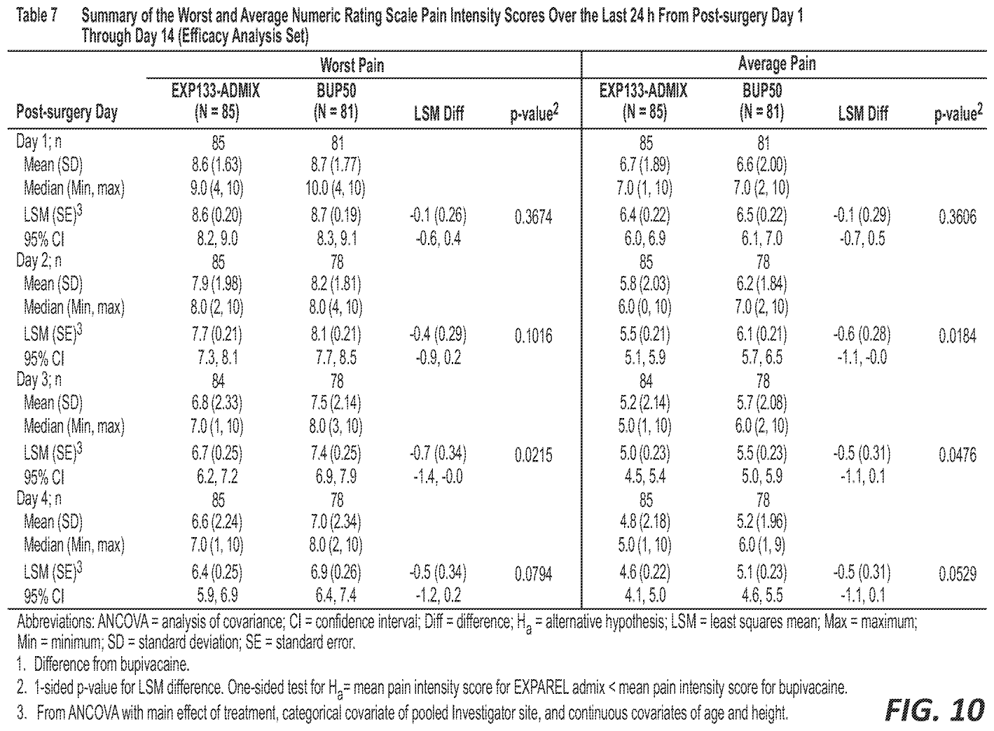

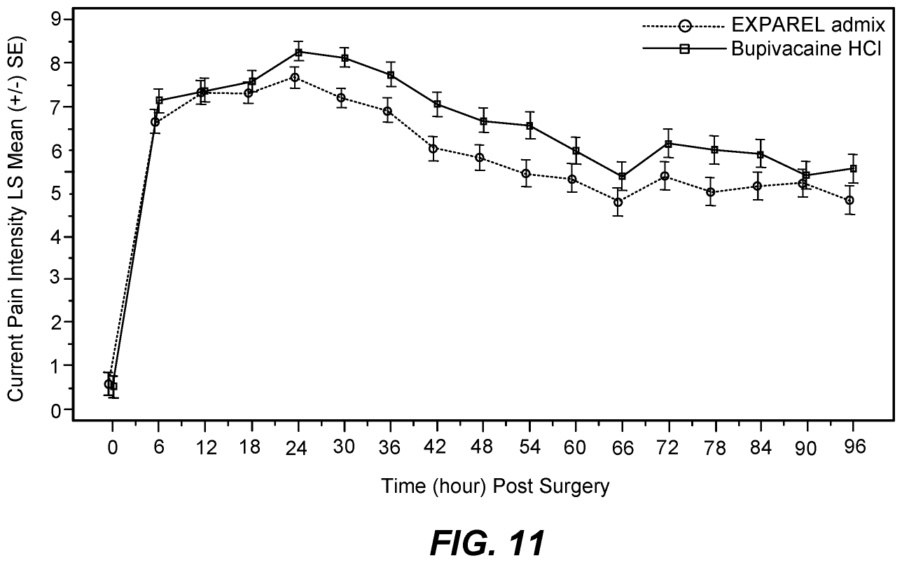

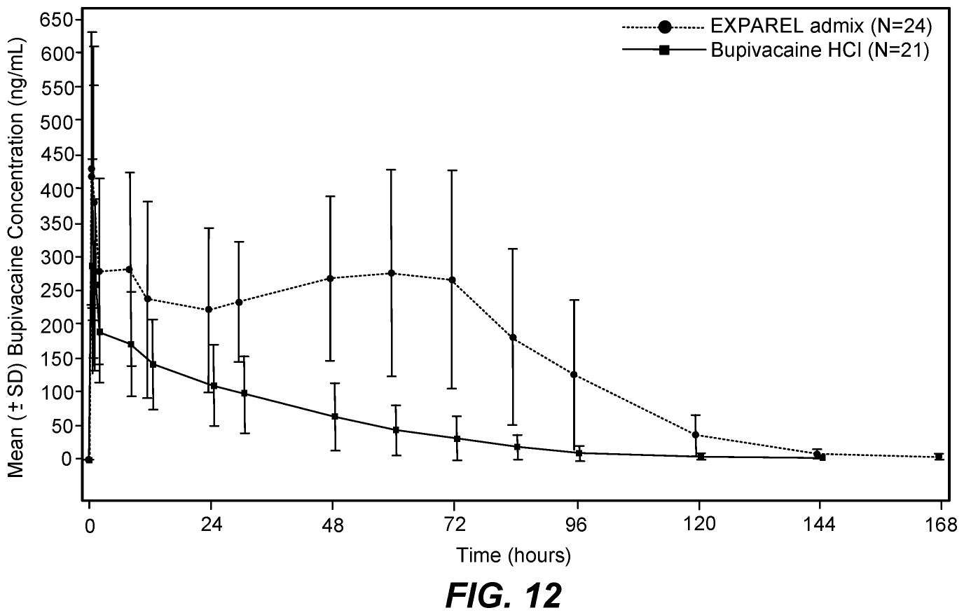

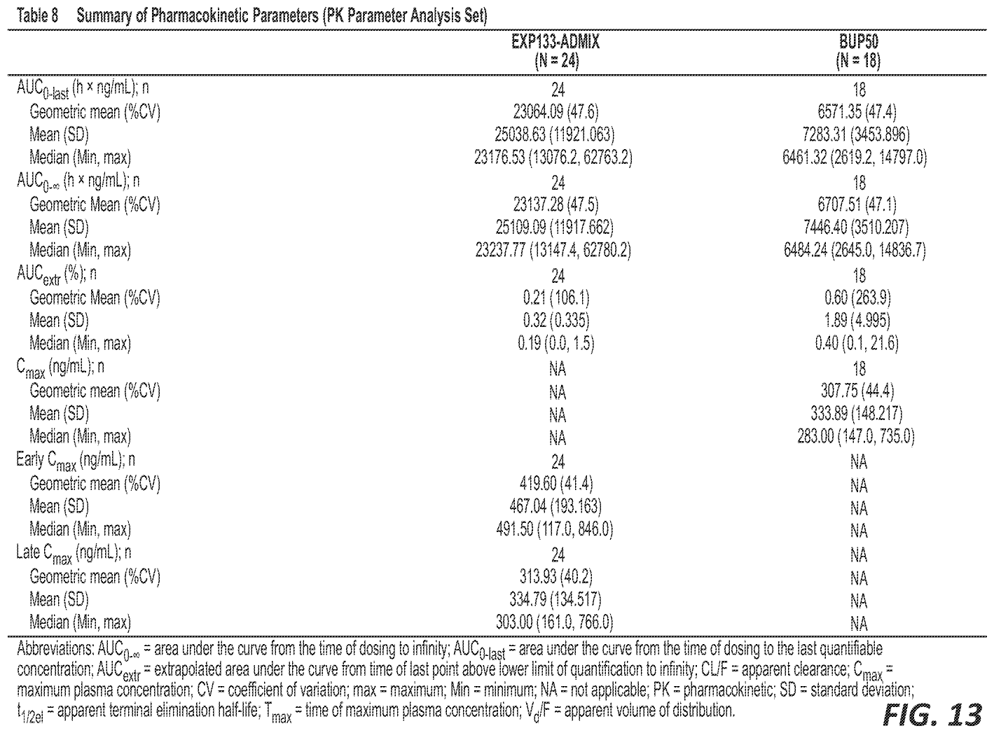

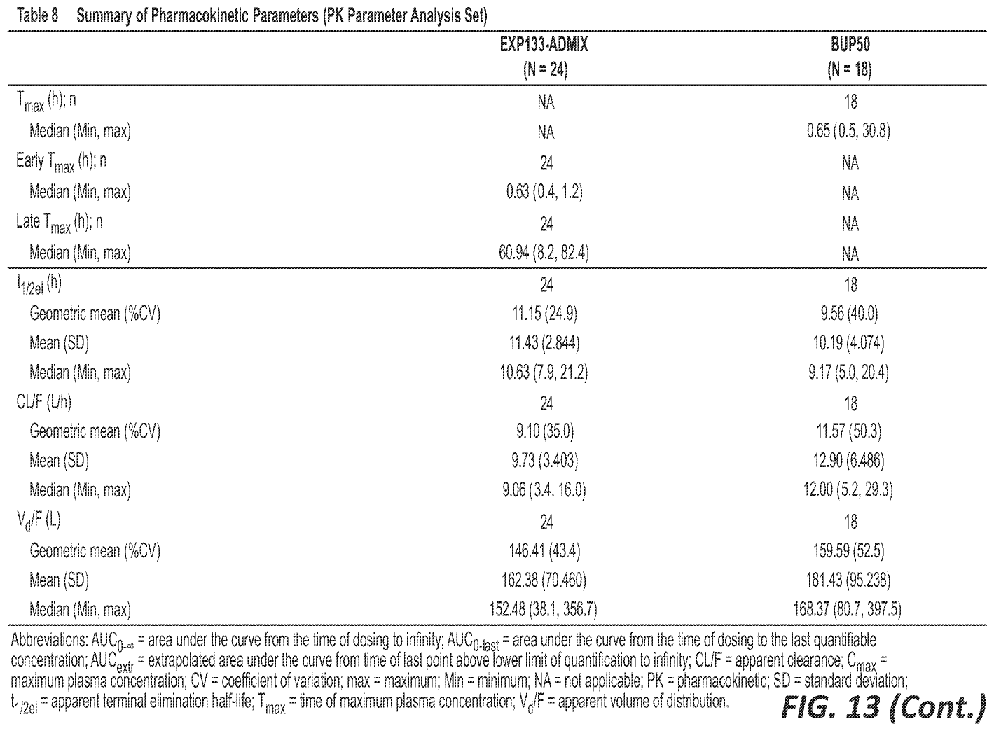

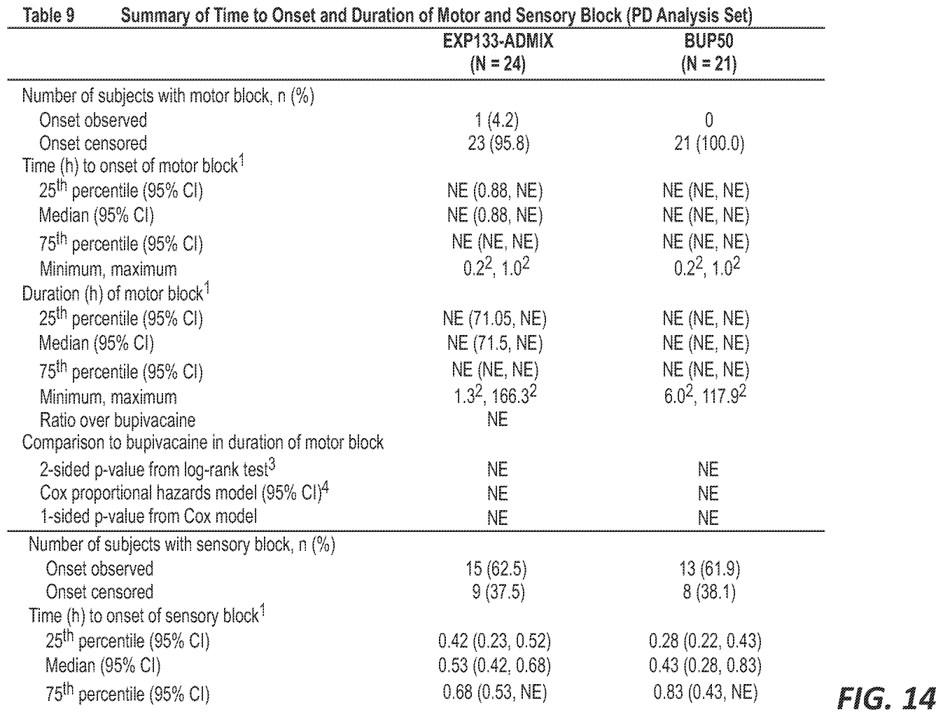

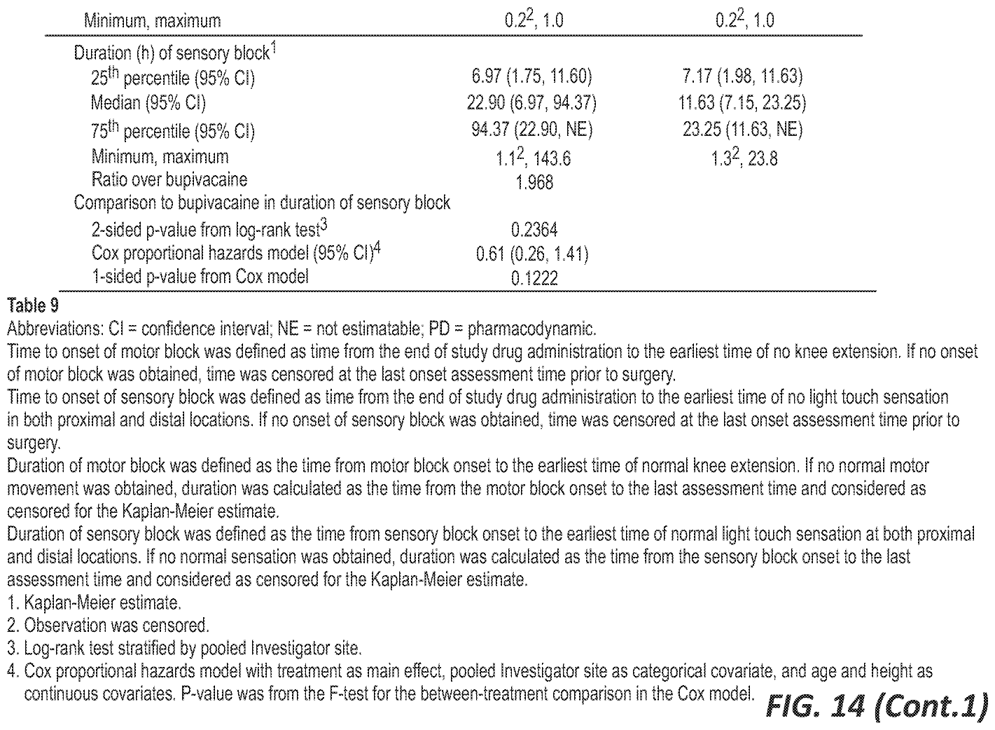

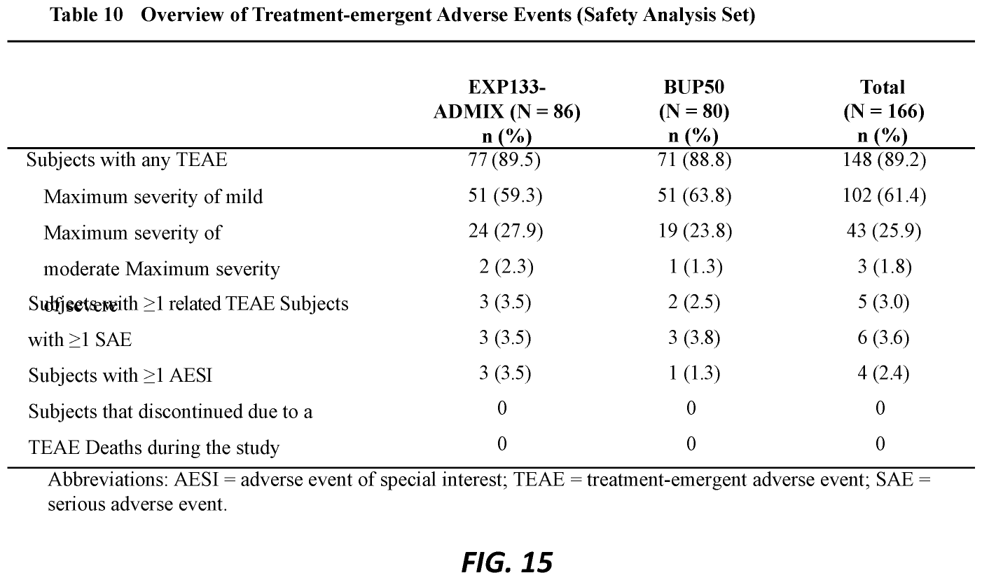

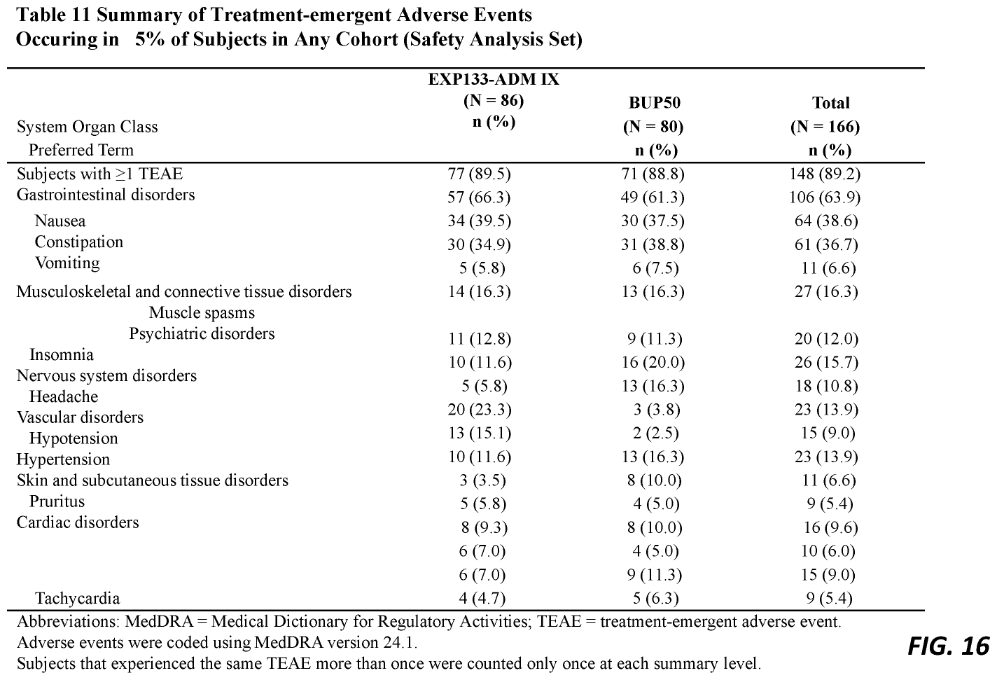

### DETAILED DESCRIPTION

Unless otherwise defined, all technical and scientific terms used herein have the same meaning as commonly understood by one of ordinary skill in the art to which this disclosure belongs. Although methods and materials similar to those described herein can be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “pain” means a physiologic and/or psychologic reaction or response to potential or actual stimulus that may result in tissue damage, injury, disease, or other condition(s). Types of pain include but are not limited to acute pain, chronic pain, thermal pain, traumatic pain, chemical pain, inflammatory pain, ischemic pain, blunt pain, sharp pain, prickling pain, visceral pain, and neuropathic pain.

### Adductor Canal/Adductor Canal Block

In some embodiments, the method includes administering a pharmaceutical composition to the adductor canal. In some embodiments, the adductor canal can be described as a conical musculoaponeurotic tunnel passing through the distal portion of the middle third of the thigh. In some embodiments, the adductor canal has three borders: the vastus medialis muscle forms the anterolateral border, the adductor longus and adductor magnus muscles form the posterolateral border, and the sartorius forms the medial wall. Major structures passing through the adductor canal include the superficial femoral artery, the femoral vein, and the saphenous nerve. The adductor canal (subsartorial canal or Hunter's canal) is about 15 cm in length and is a narrow, fascial tunnel in the thigh. The adductor canal is located deep to the middle third of the sartorius muscle. The adductor canal provides an intermuscular passage through which the femoral vessels pass to reach the popliteal fossa, where these vessels become popliteal vessels. The adductor canal begins about 15 cm (about 6 inches) inferior to the inguinal ligament, where the sartorius muscle crosses over the adductor longus muscle. The adductor canal ends at the adductor hiatus in the tendon of the adductor magnus muscle. The saphenous nerve and, in part, the obturator nerve traverse the adductor canal.

In some embodiments, administering a pharmaceutical composition to the adductor canal includes an adductor canal block. In some embodiments, administering a pharmaceutical composition to the adductor canal can be an injection into the femoral triangle. In some embodiments, administering a pharmaceutical composition to the adductor canal includes a “femoral triangle block.” In some embodiments, an adductor canal block can be used to deliver sensory anesthesia for procedures involving the distal thigh and femur, knee, and lower leg on the medial side. In some embodiments, an adductor canal block can be used to deliver sensory anesthesia for distal branches of the femoral nerve including the saphenous nerve and branches of the mixed sensory and motor nerves to the quadriceps, and branches of the obturator nerve. In some embodiments, administering a pharmaceutical composition to the adductor canal can be the administration of a sensory nerve block only, and not a motor nerve block. In some embodiments, the adductor canal is located in the middle third of the thigh. In some embodiments the adductor canal is lower on the leg than the location for a femoral nerve block. In some embodiments, the location for an adductor canal block is inferior to the location for a femoral nerve block. In some embodiments, the location for an adductor canal block is distal to the location for a femoral nerve block. In some embodiments, the location for an adductor canal block is medial to the location for a femoral nerve block.

In some embodiments, the adductor canal can be located by way of surface landmarks. In some embodiments, the adductor canal can be located by way of ultrasound guidance. In some embodiments, the adductor canal can be located by way of a combination of surface landmarks and ultrasound guidance. In some embodiments, an adductor canal block preserves quadriceps muscle strength better than a femoral nerve block. In some embodiments, an adductor canal block preserves ambulation compared to femoral nerve block. In some embodiments, an adductor canal block reduces falls compared to a femoral nerve block. In some embodiments, an adductor canal block facilitates postoperative rehabilitation compared to a femoral nerve block.

### Femoral Nerve/Femoral Nerve Block

In some embodiments, administration of a pharmaceutical composition to the adductor canal is not equivalent to administration of a pharmaceutical composition to the location for a femoral nerve block. In some embodiments, the methods of administering pharmaceutical compositions described herein, including those administering a pharmaceutical composition to the adductor canal, are not administered to location of a femoral nerve block. In some embodiments, the methods of administering pharmaceutical compositions described herein, including those administering a pharmaceutical composition to the adductor canal, do not include administering a femoral nerve block.

The anatomical location for administration of a femoral nerve block can include: identifying the inguinal ligament by drawing a line between the anterior superior iliac spine and the pubic symphysis. The femoral nerve passes through the center of this line and is at its most superficial at the level of the inguinal crease. In some embodiments, the location for a femoral nerve block is located near the hip. In some embodiments, the location for a femoral nerve block is higher on the leg than the location for an adductor canal block. In some embodiments, the location for a femoral nerve block is superior to the location of an adductor canal block. In some embodiments, the location for a femoral nerve block is proximal to the location of an adductor canal block. In some embodiments, the location for a femoral nerve block is lateral to the location of an adductor canal block.

In some embodiments, the location for a femoral nerve block can be located by way of surface landmarks. In some embodiments, the location for a femoral nerve block can be located by way of ultrasound guidance. In some embodiments, the location for a femoral nerve block can be located by way of a combination of surface landmarks and ultrasound guidance. In some embodiments, a femoral nerve block leads to a decrease in quadriceps muscle strength over an adductor canal block. In some embodiments, a femoral nerve block leads to more impaired ambulation over an adductor canal block. In some embodiments, a femoral nerve block leads to more post-operative falls than an adductor canal block. In some embodiments, a femoral nerve block requires greater postoperative rehabilitation compared to an adductor canal block.

### Pharmaceutical Compositions

Provided herein are analgesic pharmaceutical compositions. In some embodiments the pharmaceutical compositions can be used for post-operative analgesic pain.

In some embodiments, the pharmaceutical compositions include multivesicular liposomes. Multivesicular liposomes (or “MVL”, which is used herein to refer to a multivesicular liposome or a plurality of multivesicular liposomes) are lipid vesicles having multiple non-concentric internal aqueous chambers having internal membranes distributed as a network throughout the MVL. The chambers may contain acids which are effective to enable the encapsulation of bupivacaine or a salt thereof and to modulate its release rate. A preparation of MVL is described, for example, in Kim et al., Biochim. Biophys. Acta 728, 339-348, 1983. In some embodiments, a MVL is prepared in accordance with a process as described in U.S. Pat. No. 9,192,575, incorporated by reference herein in its entirety. In some embodiments, a MVL is prepared in accordance with a process as described in U.S. Pat. No. 8,182,835, incorporated by reference herein in its entirety. In some embodiments, a MVL is prepared in accordance with a process as described in U.S. Pat. No. 8,834,921, incorporated by reference herein in its entirety. In some embodiments, a MVL is prepared in accordance with a process as described in U.S. Pat. No. 9,205,052, incorporated by reference herein in its entirety.

In some embodiments the multivesicular liposomes (“MVL”) are made by the following process. A “water-in-oil” type emulsion containing a non-hydrohalic acid salt of bupivacaine, such as bupivacaine phosphate, is formed from two immiscible phases, a lipid phase and a first aqueous phase. The lipid phase is made up of at least one amphipathic lipid and at least one neutral lipid in a volatile organic solvent. The term “amphipathic lipid” refers to molecules having a hydrophilic “head” group and a hydrophobic “tail” group and may have membrane-forming capability. As used herein, amphipathic lipids include those having a net negative charge, a net positive charge, and zwitterionic lipids (having no net charge at their isoelectric point). The term “neutral lipid” refers to oils or fats that have no vesicle-forming capability by themselves, and lack a charged or hydrophilic “head” group. Examples of neutral lipids include, but are not limited to, glycerol esters, glycol esters, tocopherol esters, sterol esters which lack a charged or hydrophilic “head” group, and alkanes and squalenes.

The amphipathic lipid is chosen from a wide range of lipids having a hydrophobic region and a hydrophilic region in the same molecule. Suitable amphipathic lipids are zwitterionic phospholipids, including phosphatidylcholines, phosphatidylethanolamines, sphingomyelins, lysophosphatidylcholines, and lysophosphatidylethanolamines. Also suitable are the anionic amphipathic phospholipids such as phosphatidylglycerols, phosphatidylserines, phosphatidylinositols, phosphatidic acids, and cardiolipins. Also suitable are the cationic amphipathic lipids such as acyl trimethylammonium propanes, diacyl dimethylammonium propanes, and stearylamines.

Suitable neutral lipids are triglycerides, propylene glycol esters, ethylene glycol esters, and squalene. Examples of triglycerides useful in the present disclosure are triolein, tripalmitolein, trimyristolein, trilinolein, tributyrin, tricaproin, tricaprylin, and tricaprin. The fatty chains in the triglycerides useful in the present disclosure can be all the same, or not all the same (mixed chain triglycerides), including all different. Both saturated and unsaturated fatty chains are useful in the present disclosure. The propylene glycol esters can be mixed diesters of caprylic and capric acids.

Many types of volatile organic solvents can be used in the present disclosure, including ethers, esters, halogenated ethers, hydrocarbons, halohydrocarbons, or Freons. For example, diethyl ether, chloroform, tetrahydrofuran, ethyl acetate, Forane, and any combinations thereof are suitable for use in making the compositions of the present disclosure.

Optionally, other components are included in the lipid phase. Among these are cholesterol or plant sterols.

The first aqueous phase includes bupivacaine or a salt thereof, such as bupivacaine phosphate, at least one polyhydroxy carboxylic acid, and at least one di- or tri-protic mineral acid. In some embodiments, also included is hydrochloric acid. The di- or tri-protic mineral acids include sulfuric acid, and phosphoric acid. Also included in the first aqueous phase are such polyhydroxy carboxylic acids as glucuronic acid, gluconic acid, and tartaric acid. The di- and tri-protic mineral acids and the polyhydroxy organic acids are present in the first aqueous phase in concentrations of from 0.01 mM to about 0.5 M, or preferably from about 5 mM to about 300 mM. When hydrochloric acid is used, it is present in lower amounts, from about 0.1 mM to about 50 mM, or preferably from about 0.5 mM to about 25 mM.

The lipid phase and first aqueous phase are mixed by mechanical turbulence, such as through use of rotating or vibrating blades, shaking, extrusion through baffled structures or porous pipes, by ultrasound, or by nozzle atomization, to produce a water-in-oil emulsion. Thus, bupivacaine or a salt thereof, such as bupivacaine phosphate, is encapsulated directly in the first step of MVL manufacture.

The whole water-in-oil emulsion is then dispersed into a second aqueous phase by means described above, to form solvent spherules suspended in the second aqueous phase. The term “solvent spherules” refers to a microscopic spheroid droplet of organic solvent, within which are suspended multiple smaller droplets of aqueous solution. The resulting solvent spherules therefore contain multiple aqueous droplets with the bupivacaine or a salt thereof, such as bupivacaine phosphate, dissolved therein. The second aqueous phase can contain additional components such as glucose, and/or lysine.

The volatile organic solvent is then removed from the spherules, for instance by surface evaporation from the suspension: When the solvent is substantially or completely evaporated, MVL are formed. Gases which can be used for the evaporation include nitrogen, argon, helium, oxygen, hydrogen, and carbon dioxide. Alternatively, the volatile solvent can be removed by sparging, rotary evaporation, or with the use of solvent selective membranes.

In some embodiments, an MVL is prepared in accordance with a process as described in U.S. Pat. No. 10,398,648, incorporated by reference herein in its entirety. In some embodiments, a MVL is prepared in accordance with a process as described in U.S. Pat. No. 9,585,838 incorporated by reference herein in its entirety.

In some embodiments, an MVL is prepared in accordance with a process as described in US Published Patent Applications US 2011-0250264, US 2013-0306759, US 2013-0177634, US 2013-0177633, US 2013-0177635, US 2013-0195965, US 2013-0177636, US 2013-0183373, US 2013-0177638, US 2013-0177637, US 2013-0183372, US 2013-0183375, US 2016-0361260 or US 2018-0092847, each of which is incorporated by reference herein in its entirety.

In some embodiments, an MVL is prepared in accordance with a process as described in U.S. Pat. Nos. 11,033,495; 11,179,336; 11,278,494; 11,304,904; 11,311,486; 11,357,727; 11,426,348; 11,452,691, each of which is incorporated by reference herein in its entirety.

In some embodiments, the pharmaceutical compositions described herein can be combined, used in conjunction with, or used in an anesthetic or analgesic program with other anesthetics or analgesics.

Examples of anesthetics, include but are not limited to, propofol, etomidate, methohexital and sodium thiopental, midazolam, diazepam, and ketamine, benzocaine, chloroprocaine, cocaine, cyclomethycaine, dimethocaine, propoxycaine, procaine, proparacaine, tetracaine, articaine, bupivacaine, carticaine, dibucaine, etidocaine, levobupivacaine, lidocaine, mepivacaine, piperocaine, prilocaine, ropivacaine, trimecaine, saxitoxin, and tetrodotoxin. Examples of amide anesthetics, include but are not limited to, articaine, bupivacaine, carticaine, dibucaine, etidocaine, levobupivacaine, lidocaine, mepivacaine, piperocaine, prilocaine, ropivacaine, and trimecaine. In some embodiments, the multivesicular liposomes further comprise bupivacaine, morphine, cytarabine, or their pharmaceutically acceptable salts as the therapeutic agent. In some embodiments, the multivesicular liposomes further comprise bupivacaine phosphate, morphine sulfate, or cytarabine HCl.

The term “therapeutically effective” as it pertains to bupivacaine or a salt thereof, such as bupivacaine phosphate, present in the pharmaceutical compositions described herein, means that an anesthetic present in the first aqueous phase within the multivesicular liposome is released in a manner sufficient to achieve a particular level of anesthesia. Exact dosages will vary depending on such factors as the particular anesthetic, as well as patient factors such as age, sex, general condition, patient size, and the like. Those of skill in the art can readily take these factors into account and use them to establish effective therapeutic concentrations without resort to undue experimentation.

As used herein, “non-liposomal bupivacaine” refers to bupivacaine or a salt thereof that is not in liposomal form. For example, “non-liposomal bupivacaine” refers to bupivacaine or a salt thereof that is not comprised in a multivesicular liposome. The term “non-liposomal bupivacaine” encompasses compositions comprising bupivacaine, or a salt thereof, that is not in liposomal form.

Examples of analgesics can include opioid analgesics and non-opioid analgesics. Non-limiting examples of opioid analgesics include hydrocodone, oxycodone, propoxyphene, or fentanyl, thiosemicarbazone, p-nitrophenylhydrazone, o-methyloxime, semicarbazone, or bis (methylcarbamate), oxycodone, a pharmaceutically acceptable salt or its thiosemicarbazone, p-nitrophenylhydrazone, o-methyloxime, semicarbazone, or bis-methylcarbamate. Non-limiting examples of non-opioid analgesics useful in the present invention include aspirin; acetaminophen; a non-steroidal anti-inflammatory drug (NSAID), an arylalkanoic acid, a profen, a fenamic acid, an oxicam, a pyrazolidine derivative; a Cox-2 inhibitor, a local analgesic, an anti-depressant, an atypical analgesic, a psychotropic agent, an NMDA receptor antagonist, an α2-adrenoreceptor agonists and a synthetic drug having narcotic properties.

Embodiments of the present disclosure also include compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Academic Press, (Adeboye Adejareedit edit., 2020), hereby incorporated by reference in its entirety.

A pharmaceutically effective dose is that dose required to prevent, inhibit the occurrence, or treat (alleviate a symptom to some extent, preferably all of the symptoms) pain. One skilled in the art appreciates that compositions and methods of the present disclosure can be used to treat multiple types of pain, and that the effective dose may be different for different types of pain. Types of pain include but are not limited to thermal pain, chemical pain, inflammatory pain, ischemic pain, trauamatic pain, blunt pain, sharp pain, prickling pain, and visceral pain. The pharmaceutically effective dose depends on the type of condition (e.g., pain), the composition used, the route of administration, the type of mammal being treated, the physical characteristics of the specific mammal under consideration (including but not limited to age, physical condition, surgical or other medical procedures being performed, circulatory capacity, cardiovascular function, pain tolerance, nerve function, liver function), concurrent medication, and other factors that those skilled in the medical arts will recognize.

### Methods of Administering

Provided herein are methods of administering an adductor canal block. In some embodiments, the methods include administering to the adductor canal of a patient a pharmaceutical composition for post-operative analgesia, comprising: (a) selecting an entry point of an injection needle in a leg of a patient by ultrasound transducer; (b) inserting the injection needle into the patient at the entry point; (c) identifying a first nerve in the leg of the patient; (d) administering to the first nerve saline and a pharmaceutical composition; (e) identifying a second nerve in the leg of the patient; (f) administering to the second nerve saline and the pharmaceutical composition; wherein the first nerve and second nerve are selected from the group consisting of the nerve to vastus medialis (NVM) and the saphenous nerve, and wherein the first nerve is not the second nerve, and wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome, thereby administering to the adductor canal of the patient the pharmaceutical composition for post-operative analgesia.

In some embodiments, the methods include administering to the adductor canal of a human patient a pharmaceutical composition for post-operative analgesia, comprising: (a) selecting an entry point of an injection needle in a leg in a patient by ultrasound transducer; (b) advancing a needle tip of the injection needle within a region between a vastus medialis muscle and a sartorius muscle of the patient along a trajectory that extends between the entry point and a superficial femoral artery; (c) identifying a first nerve in the leg of the patient; (d) administering to the first nerve through the injection needle saline and about 10 mL of a pharmaceutical composition; (e) identifying a second nerve in the leg of the patient; (f) administering to the second nerve through the injection needle saline and about 10 mL of the pharmaceutical composition; wherein the first nerve and second nerve are selected from the group consisting of the nerve to vastus medialis (NVM) and the saphenous nerve, and wherein the first nerve is not the second nerve, and wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome, thereby administering to the adductor canal of the human patient the pharmaceutical composition for post-operative analgesia.

In some embodiments, the methods include treating post-operative knee pain in a patient, comprising: (a) selecting an entry point of an injection needle in a leg of the patient; (b) inserting the injection needle into the leg of the patient at the entry point; (c) identifying a first nerve in the leg of the patient; (d) administering to the first nerve saline and a pharmaceutical composition; (e) identifying a second nerve in the leg of the patient; (f) administering to the second nerve saline and the pharmaceutical composition; wherein the first nerve and second nerve are selected from the group consisting of the nerve to vastus medialis (NVM) and the saphenous nerve, and wherein the first nerve is not the second nerve, and wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome, thereby treating post-operative knee pain in the patient.

In some embodiments, the methods include, administering an adductor canal block to a patient, comprising: (a) selecting an entry point of an injection needle in a leg of a patient by ultrasound transducer, wherein the entry point comprises a region adjacent to a superficial femoral artery in the middle of a sartorius muscle; (b) advancing a needle tip of the injection needle within a region between a vastus medialis muscle and a sartorius muscle of the patient along a trajectory that extends between the entry point and a superficial femoral artery; (c) identifying a nerve to vastus medialis (NVM) in the patient; (d) administering to the NVM through the injection needle saline and about 10 mL of a multivesicular liposome pharmaceutical composition; (e) identifying a saphenous nerve, wherein identifying a saphenous nerve in the patient comprises penetrating a vasto-adductor membrane with the needle tip and advancing the needle tip to a location anterior to the superficial femoral artery; (f) administering to the saphenous nerve through the injection needle saline and about 10 mL of a multivesicular liposome pharmaceutical composition to the saphenous nerve; wherein the multivesicular liposome pharmaceutical composition comprises: bupivacaine or a salt thereof; phosphoric acid; a lipid component comprising at least one amphipathic lipid and at least one neutral lipid lacking a hydrophilic head group; and, optionally, a cholesterol and/or a plant sterol, thereby administering the adductor canal block to the patient.

Further, these methods can also be administered to a patient for treating knee pain associated with total knee arthroplasty, reducing an amount of post-operative pain in a patient following total knee arthroplasty surgery, reducing an amount of an opioid analgesic administered to a patient following total knee arthroplasty surgery, administering to the adductor canal of a patient a pharmaceutical composition, or administering to the adductor canal of a patient a pharmaceutical composition for the management of post-operative pain associated with total knee arthroplasty.

### Routes of Administration

Anesthetics of the present disclosure may be delivered regionally or locally. “Regional” or “local” anesthesia, as used herein, is distinct from general anesthesia and refers to anesthetic procedures which allow for the preferential delivery of an anesthetic to a specific region of the body, such as near a nerve or a nerve bundle. In contrast, general anesthesia allows for the systemic administration of an anesthetic, e.g., via intravenous administration. Regional or local anesthesia typically allows for a lower total body concentration (although elevated local concentrations) of an anesthetic to be administered to a subject for analgesia or diminished pain perception of at least a portion of the subject's body. For example, intrathecal anesthesia, epidural anesthesia, and nerve blocks are examples of regional or local anesthesia.

A pharmacological composition can refer to a composition in a form suitable for administration, e.g., perineural administration, into a subject or proximal to at least one nerve of a subject, including for example wherein the subject is a human. Suitable forms, in part, depend upon the use or the route of entry. Examples of routes of entry include but are not limited to injection (including but not limited to subcutaneous injection), single injection, serial injection, indwelling catheter, and continuous infusion. Such routes of entry should not prevent the composition from reaching a target cell (i.e., a neuron). For example, injectable pharmacological compositions should be soluble. Other factors are known in the art, and include considerations such as toxicity and forms that prevent the composition or formulation from exerting its effect.

### EXAMPLES

### Example 1—Clinical Trial #1

A multicenter, randomized, double-blind clinical trial examining efficacy and safety of the pharmaceutical composition comprising: a) the multivesicular liposome disclosed herein and b) the aqueous phase disclosed herein, wherein the aqueous phase is encapsulated within the multivesicular liposome, was performed as described below. The pharmaceutical composition is referred to below as the “multivesicular liposomal” pharmaceutical composition, or “MVL”.

The trial compared the magnitude of the postsurgical analgesic effect following a single dose of the MVL admixed with bupivacaine HCl vs. bupivacaine HCl when administered via an adductor canal block in subjects undergoing primary unilateral TKA. Further, the trial compared post-surgical opioid consumption following a single dose of MVL admixed with bupivacaine HCl vs. bupivacaine HCl. Additionally, the trial compared the time to first opioid consumption post-surgery, following a single dose of MVL admixed with bupivacaine HCl vs. bupivacaine HCl. Additionally, the trial characterized and compared the magnitude of the duration of sensory and motor block following a single dose of MVL admixed with bupivacaine HCl and bupivacaine HCl. Further, the trial assessed the safety and PK profile of MVL admixed with bupivacaine HCl and bupivacaine HCl.

| Abbreviation/ | |
|---|---|
| Term | Definition |
| λ_{z} | Apparent terminal elimination constant rate |
| AE | Adverse event |
| AESI | Adverse event of special interest |
| ANCOVA | Analysis of covariance |
| ASA | American Society of Anesthesiologists |
| AUC | Area under the curve |
| AUC_{0-∞} | Area under the curve from the time of dosing to infinity |
| AUC₀₋ₗₐₛₜ | Area under the curve from the time of dosing to the last |
| | quantifiable concentration |
| AUCₑₓₜᵣ | Extrapolated area under the curve from time of last point |
| | above lower limit of quantification to infinity |
| BLOQ | Below the limit of quantification |
| BMI | Body mass index |
| CFR | Code of Federal Regulations |
| CI | Confidence interval |
| CL/F | Apparent clearance |
| Cₘₐₓ | Maximum plasma concentration |
| Cₜ | The last quantifiable concentration |
| CV | Coefficient of variation |
| Diff | Difference |
| eCRF | Electronic case report form |
| EKG | Electrocardiogram |
| FDA | Food and Drug Administration |
| GCP | Good Clinical Practice |
| h | Hour(s) |
| Hₐ | Alternative hypothesis |
| HCF | Health care facility |
| HCl | Hydrochloride |
| ICF | Informed consent form |
| ICH | International Council for Harmonisation |
| ID | Identification |
| IEC | Independent Ethics Committee |
| Intra-op | During operation |
| IPACK | Infiltration between the popliteal artery and the posterior |
| | capsule of the knee |
| IPO | International Pain Outcome |
| IRB | Institutional Review Board |
| IV | Intravenous(ly) |
| LLOQ | Lower limit of quantification |
| LSM | Least squares (mean) |
| Max | Maximum |
| MedDRA | Medical Dictionary for Regulatory Activities |
| Min | Minimum |
| min | Minute(s) |
| NA | Not applicable |
| NCA | Noncompartmental analysis |
| NE | Not estimatable |
| NRS | Numeric Rating Scale |
| NSAID | Non-steroidal anti-inflammatory drug |
| OMED | Oral morphine equivalents |
| OR | Operating Room |
| PACU | Post-anesthesia Care Unit |
| PD | Pharmacodynamic |
| PK | Pharmacokinetic |
| PO | Orally administered |
| POD | Post-operative day |
| Post-op | Post-operative |
| Pre-op | Pre-operative |
| PT | Preferred term |
| Reduct | Reduction |
| SAE | Serious adverse event |
| SAP | Statistical Analysis Plan |
| SD | Standard deviation |
| SE | Standard error |
| SOC | System organ class |
| t_{1/2el} | Apparent terminal elimination half-life |
| TEAE | Treatment-emergent adverse event |
| TKA | Total knee arthroplasty |
| Tₘₐₓ | Time of maximum plasma concentration |
| V_{d}/F | Apparent volume of distribution |
| WOCBP | Women of childbearing potential |

### Inclusion Criteria

Each subject had to meet the following criteria to be eligible for the study:
1. Male or female, ages 18 or older at screening.
2. Indicated to undergo primary unilateral TKA under spinal anesthesia.
3. Primary indication for TKA was degenerative osteoarthritis of the knee.
4. American Society of Anesthesiologists (ASA) physical status 1, 2, or 3 (see Section 18.5 of the Protocol [Section 16.1.1]).
5. Able to provide informed consent, adhere to the study schedule, and complete all study assessments.
6. Body mass index≥18 and <40 kg/m2.

### Exclusion Criteria

Subjects who met any of the following criteria were excluded from the study:
1. Allergy, hypersensitivity, intolerance, or contraindication to any of the study medications for which an alternative was not named in the Protocol (e.g., amide-type local anesthetics, opioids, bupivacaine HCl, non steroidal anti inflammatory drugs [NSAIDs]).
2. Planned concurrent surgical procedure (e.g., bilateral TKA).
3. Undergoing unicompartmental TKA or revision TKA.
4. Concurrent painful physical condition (e.g., arthritis, fibromyalgia, cancer) that may have required analgesic treatment with NSAIDs or opioids in the post dosing period for pain that were not strictly related to the knee surgery and which, in the Investigator's opinion, may have confounded the post dosing assessments.
5. Inadequate sensory function below the knee as assessed by the Investigator.
6. History of contralateral TKA within 1 year.
7. Previous open knee surgery on the knee being considered for TKA. Prior arthroscopy was permitted.
8. History of, suspected, or known addiction to or abuse of illicit drug(s), prescription medicine(s), or alcohol within the past 2 years.
9. Administration of an investigational drug within 30 days or 5 elimination half-lives of such investigational drug, whichever was longer, prior to study drug administration, or planned administration of another investigational product or procedure during the subject's participation in this study.
10. Previous participation in an EXPAREL study.
11. Uncontrolled anxiety, schizophrenia, or other psychiatric disorder that, in the opinion of the Investigator, could have interfered with study assessments or compliance.
12. Currently pregnant, nursing, or planning to become pregnant during the study.
13. Clinically significant medical disease that, in the opinion of the Investigator, would have made participation in a clinical study inappropriate. This included diabetic neuropathy, coagulation or bleeding disorders, severe peripheral vascular disease, renal insufficiency, hepatic dysfunction or other conditions that would have constituted a contraindication to participation in the study.
14. Currently on a neuromodulating agent (e.g., gabapentin, pregabalin [Lyrica®], duloxetine [Cymbalta®], etc.)].
15. Current use of systemic glucocorticoids within 30 days of randomization in this study.
16. Use of dexmedetomidine HCl (Precedex®) or clonidine within 3 days of study drug administration.
17. Any use of marijuana (including tetrahydrocannabinol and cannabidiol) within 30 days prior to randomization, or planned use during the course of the study.
18. Chronic opioid use (average ≥30 OMED/day) within 30 days prior to randomization.

### Methods

This was a Phase 3, multicenter, randomized, double blind, active controlled study of 167 subjects undergoing primary unilateral TKA under spinal anesthesia. The study consisted of 2 cohorts that were enrolled in parallel. Cohort 1 enrolled 46 subjects to obtain information on the PK profile, pharmacodynamics (PD), efficacy, and safety. Cohort 2 enrolled 121 subjects to obtain information on efficacy and safety. An adaptive study design was used in this study, with an interim analysis conducted by an unblinded independent review committee to evaluate sample size assumptions and futility.

Subject participation began upon obtaining informed consent, which was obtained within ≤45 days prior to administration of the study drug. Screening procedures included assessment of eligibility; recording of medical/surgical history, prior and concomitant medications (related to medical history), demographics and baseline characteristics, and height and weight for body mass index (BMI) calculation; assessment of chronic opioid and any Cannabis use in the past 30 days (average ≥30 OMED/day); urine pregnancy test for women of childbearing potential (WOCBP); 12-lead electrocardiogram (EKG); and monitoring of adverse events (AEs) and serious adverse events (SAEs).

On the day of surgery, prior to surgery, subjects in Cohort 1 and Cohort 2 were randomized (1:1) to receive an adductor canal block with a single dose of either 10 mL EXPAREL (133 mg) admixed with 10 mL 0.5% bupivacaine HCl (50 mg; from hereon referred to as the EXP133-ADMIX arm) or 10 mL 0.5% bupivacaine HCl (50 mg) mixed with 10 mL normal saline (from hereon referred to as the BUP50 arm). The total dose volume was consistent (20 mL) for all subjects.

Subjects may have been lightly sedated with 1 to 2 mg of intravenous (IV) midazolam before the nerve block procedure. The study drug was administered under ultrasound guidance 90 (±30) min prior to surgery. A peripheral nerve stimulator was used to confirm nerves in the adductor canal before study drug administration. A confirmatory ultrasound video was captured during the nerve block procedure (during hydrodissection by saline injection and study drug administration), with needle in place to ensure accurate block placement. All subjects in both Cohort 1 and Cohort 2 received an infiltration between the popliteal artery and capsule of the knee under ultrasound guidance with 15 mL of 0.25% bupivacaine HCl (37.5 mg) immediately following study drug administration (IPACK infiltration procedure).

Treatment prior to study drug administration may include Celecoxib 200 mg orally administered within 4 hours prior to surgery. Other permitted prior medications and therapy may include 1 to 2 mg of midazolam and/or ondansetron. Restricted medications and therapy prior to drug study administration included systemic glucocorticosteroids and neuromodulating agents (e.g., gabapentin, pregabalin [Lyrica®], duloxetine [Cymbalta®], etc.); no long-acting or sustained release opioid medications and NSAIDs (except for low-dose acetylsalicylic acid used for cardiovascular protection) within 3 days of study drug administration; no dexmedetomidine HCl (Precedex®) or clonidine within 3 days of study drug administration; no scopolamine patches; no opioid medications within 24 hours of study drug administration; no use of an investigational product within 30 days or 5 elimination half-lives of such investigational drug, whichever was longer, prior to study drug administration, or planned administration of another investigational product or procedure during the subject's participation in this study was not permitted; no drugs (other than the described bupivacaine HCl admixture) were to be admixed with study drug (e.g., epinephrine, dexamethasone, clonidine); no lidocaine or other local anesthetics were locally administered in the area of the nerve block administration other than use in a superficial cutaneous wheal for needle insertion.

Perioperative treatment may include receiving an IPACK infiltration under ultrasound guidance with 15 mL (37.5 mg) 0.25% bupivacaine HCl immediately following study drug administration (was to be done with the same set-up for the nerve block); receiving spinal anesthesia immediately prior to surgery with 0.5% bupivacaine HCl (≥15 mg). If the spinal failed or could not be completed, the subject may have received total intravenous anesthesia; receiving 1000 mg of IV acetaminophen at the time of surgical incision; receiving propofol for induction and intra-operative sedation. Other permitted medications may include tranexamic acid. Restricted medications included no other medication (including opioids) were to be mixed with the bupivacaine for spinal anesthesia; no use of dexamethasone, acetaminophen/paracetamol, ketorolac, or other NSAIDs preemptively or intra operatively, except for emergency use to treat an AE; no intra operative use of opioids (except IV fentanyl at a dose not exceeding 1 μg/kg, unless deemed medically necessary) and ketamine.

Post-surgical treatment may include receiving 1 post-operative dose of 1000 mg IV acetaminophen, administered approximately 8 hours after the first dose (approximately 8 hours after incision), maximum total dose was not to exceed 2000 mg; no additional acetaminophen was permitted after the second IV acetaminophen dose. Other permitted medications may include ondansetron or metoclopramide could be used for postoperative nausea and vomiting. Restricted medications included no other analgesics, including fentanyl, within 96 hours after surgery, no scopolamine patches; no patient controlled analgesia; no dexmedetomidine HCl (Precedex®) use; no lidocaine (except, if used as a local anesthetic at the site of IV placement) or other local anesthetics to be locally administered in the area of the nerve block administration through POD7; systemic glucocorticosteroids and neuromodulating agents (e.g., gabapentin, pregabalin [Lyrica®], duloxetine [Cymbalta®], etc.).

An unscheduled pain intensity assessment using the NRS (measured as “On a scale from 0 to 10, where 0 equals no pain and 10 equals the worst possible pain, how much pain are you experiencing in your operative knee right now?”) was to be completed immediately prior to administration of any breakthrough pain medication up to 96 hours post-surgery. Medications were administered as needed; opioids were not to be given on a pre-determined schedule. Immediate release PO oxycodone could be administered in a stepwise approach as follows: initial dose of 5 mg oxycodone offered; if the initial opioid dose was insufficient for pain relief, an additional 5 mg oxycodone could be offered up to a maximum of 10 mg (total dose); if a subject was unable to tolerate PO medication or the PO oxycodone pain relief was insufficient, IV morphine (initiated at 2 mg) or hydromorphone (initiated at 0.2 mg) could be administered. No NSAIDs or other opioids, including tramadol were allowed for breakthrough pain management. No acetaminophen (other than the scheduled IV acetaminophen) was to be used for breakthrough pain. Pain management modalities were standardized during the first 96 hours post-surgery. After 96 hours, the analgesic regimen could be adjusted for each subject individually as deemed appropriate by the physician responsible for the postsurgical care.

All postsurgical analgesics administered, were to be recorded through hospital discharge.

Efficacy assessments included pain intensity scores focused on the operative knee using the Numeric Rating Scale (NRS), total postsurgical opioid consumption in OMED, time to first opioid consumption post-surgery, and subject satisfaction using the International Pain Outcome (IPO). Additionally, blood samples for PK analysis (area under the curve [AUC], maximum plasma concentration [Cmax], time of maximum plasma concentration [Tmax], including early and late Cmax and Tmax (EXP133-ADMIX arm only), apparent terminal elimination half-life [t½e1], apparent clearance [CL/F], and apparent volume of distribution [Vd/F]) and PD assessment data (sensory and motor block) were collected from Cohort 1 subjects.

Safety assessments included monitoring of AEs (including SAEs and adverse events of special interest [AESIs]), vital signs (temperature, resting heart rate, respiratory rate, oxygen saturation, and blood pressure), 12 lead EKG, and recording of concomitant medications.

The Screening Visit was conducted within 45 days prior to study drug administration and the time from study drug administration until the end of participation was post-operative day (POD) 14 (±3 days), at which time a final follow up telephone call occurred. Therefore, the total duration of participation in the study for subjects was up to 62 days.

### End Points

The primary endpoint was the AUC of the NRS pain intensity scores from 0 to 96 hours post-surgery.

Secondary efficacy endpoints included total postsurgical opioid consumption in OMED from 0 to 96 hours post-surgery; time to first opioid consumption post-surgery; and worst and average NRS pain intensity scores at 24, 48, 72, and 96 hours post-surgery.

Exploratory efficacy endpoints included opioid-free status from 0 to 24, 48, 72, and 96 hours post-surgery; subject satisfaction as measured by IPO at 96 hours post-surgery; AUC of “average” pain scores from POD1 through POD4; AUC of “worst” pain scores from POD1 through POD4; and current pain intensity scores from 0 to 96 hours post-surgery.

Safety endpoints included incidence of treatment emergent adverse events and SAEs from start of the nerve block procedure through POD14; change from baseline in vital signs over time; and abnormal post-baseline EKG findings.

Pharmacokinetic endpoints included area under the plasma concentration-versus-time curve, specifically from the time of dosing to the time of the last quantifiable concentration (AUC0-last) and AUC from the time of dosing to infinity (AUC0-∞); maximum plasma concentration (Cmax) and time of maximum plasma concentration (Tmax) for subjects treated with bupivacaine HCl; early and late Cmax and Tmax were determined for subjects treated with EXPAREL admixed with bupivacaine HCl (EXP133-ADMIX arm); t½e1; CL/F; Vd/F.

Pharmacodynamic endpoints included: duration of the sensory block and motor block; time to onset of sensory block and motor block; and time to offset of sensory block and motor block.

Pharmacokinetic Parameters

| Parameter | Definition |
|---|---|
| Cₘₐₓ¹ | The maximum observed plasma concentration obtained |
| | directly from the concentration-time data |
| Early | The maximum observed plasma concentration occurring |
| Cₘₐₓ² | between dosing (0 h) and x h after dosing if appropriate |
| | based on the individual subject and treatment arm mean |
| | concentration-time plots, where x was determined from |
| | the concentration-time plot |
| Late | The maximum observed plasma concentration occurring |
| Cₘₐₓ² | more than x h after dosing if appropriate based on the |
| | individual subject and treatment arm mean concentration- |
| | time plots, where x was a subject-specific local minimum |
| | turning point in the concentration-time curve |
| Tₘₐₓ¹ | Time at which Cₘₐₓ was observed |
| Early | Time at which early Cₘₐₓ was observed |
| Tₘₐₓ² | |
| Late | Time at which late Cₘₐₓ was observed |
| Tₘₐₓ² | |
| λ_{z} | The apparent terminal elimination rate constant was |
| | estimated at the terminal phase by linear regression after |
| | log-transformation of the concentrations |
| t_{1/2el} | The apparent terminal elimination half-life was calculated |
| | as ln(2)/λ_{z} |
| Cₜ | The last quantifiable concentration |
| AUC₀₋ₗₐₛₜ | The area under the plasma concentration-time curve from |
| | the time of dosing to the time of the last quantifiable |
| | concentration; calculated using the linear-up/log-down |
| | trapezoidal method |
| AUC_{0-∞} | The area under the plasma concentration-time curve from |
| | the time of dosing (zero) to infinity; calculated as the sum of |
| | AUC₀₋ₗₐₛₜ and residual area Cₜ/λ_{z} |
| AUCₑₓₜᵣ | Extrapolated area under the curve from time of last point |
| | above LLOQ to infinity, expressed as percentage of AUC_{0-∞}; |
| | calculated as (Cₜ/λ_{z})/AUC_{0-∞} × 100%. |
| CL/F | Apparent clearance was estimated as dose/AUC_{0-∞}, with |
| | doses calculated as follows (note that every subject received |
| | 37.5 mg bupivacaine HCl during IPACK): |
| | EXP133-ADMIX arm = 133 + ([50 + 37.5] × 0.8867) = |
| | 210.6 mg |
| | BUP50 arm = (50 + 37.5 mg) × 0.8867 (salt to free base |
| | conversion) = 77.6 mg |
| V_{d}/F | Apparent volume of distribution was estimated as (CL/F)/λ_{z} |

Abbreviations: HCL = hydrochloride; IPACK = infiltration between the popliteal artery and capsule of the knee; LLOQ = lower limit of quantitation.
¹Calculated for BUP50 arm only.
²Calculated for EXP133-ADMIX arm only.

### Statistical Analysis

Descriptive statistics (number of subjects, mean, standard deviation [SD], median, minimum, and maximum) were provided for continuous data. Tabulations (number and percentage of subjects) by category were provided for categorical data. Unless specified otherwise, all confidence intervals (CIs) were 2-sided with 95% confidence. All hypothesis tests were between the select EXPAREL dose group and the bupivacaine HCl group. All statistical comparisons were 1-sided tests at an alpha level of 0.025.

The AUC of NRS pain intensity scores from 0-96 hours post-surgery were analyzed using the analysis of covariance (ANCOVA) model. The main contrast of interest to assess treatment effect was the difference between treatment arms in the AUC of pain score's least square means.

Total postsurgical opioid consumption in OMED from 0 to 96 hours was analyzed using an ANCOVA model. Time to first postsurgical opioid medication was analyzed using the Kaplan Meier survival method. Worst and average NRS pain intensity scores through 24 h, 48 h, 72 h, and 96 hours from the end of surgery were summarized by treatment arm.

To control for the overall Type-I error rate for the multiple comparisons in the efficacy analyses, the statistical tests were conducted in a hierarchical order as follows:
1. Primary endpoint (AUC of NRS pain 0 to 96 hours post-surgery).
2. First secondary endpoint (Total postsurgical opioid dose 0 to 96 hours).
3. Second secondary endpoint (Time to first postsurgical opioid).
4. Third secondary endpoint (worst and average NRS pain intensity scores at 24, 48, 72, and 96 hours post-surgery).

At any step, if the statistical test became non-significant, all the subsequent tests were deemed non-significant.

Adverse events and SAEs were recorded from the time of informed consent through POD14. Adverse events were coded using the Medical Dictionary for Regulatory Activities version 24.1 and summary tables were based on the Safety Analysis Set. A subject data listing was provided for all AEs, which included the reported term, preferred term, system organ class, treatment-emergent adverse event flag, study day of AE onset, AE start/stop date and time, relationship to study drug, frequency, severity, action taken with subject, outcome, and seriousness criteria.

Vital signs and their change from baseline were summarized with mean, median, and standard deviation at baseline and at each scheduled time point. The frequency and percentage of EKG findings (normal, abnormal/not clinically significant, and abnormal/clinically significant) were summarized at screening and each scheduled time point.

An interim analysis was conducted to evaluate sample size assumptions and futility. The interim analysis was conducted when a total of 80 subjects (40 subjects in each arm) combined from either Cohort 1 or Cohort 2 were enrolled and provided complete assessment data for the primary efficacy outcome.

### Results

Efficacy
From 0 to 96 hours post-surgery, subjects in the EXP133 ADMIX arm had a lower mean AUC of NRS Pain Intensity Score compared with the BUP50 arm with an LSM difference of −65.8 (95% CI=−118.7, −12.9; p=0.0074). The EXP133 ADMIX arm had significantly lower mean AUC of NRS Pain Intensity Scores than the BUP50 arm, particularly after the first 24 hours post-surgery (LSM difference range=−55.9 to 10.0; p values ≤0.0404).
For all time intervals in the first 96 hours post-surgery, subjects in the EXP133 ADMIX arm had a lower total opioid consumption compared with the BUP50 arm with LSM ratios over bupivacaine ranging from 0.72 to 0.83; this was particularly evident in the first 48 hours post-surgery (p-values ≤0.0201).
All subjects took opioid rescue medication. The median time to first rescue medication was 4.15 hours (95% CI=3.80, 4.83) for the EXP133-ADMIX arm and 3.63 hours (95% CI=2.98, 4.05) for those in the BUP50 arm, with a hazard ratio of 0.70 (95% CI=0.51, 0.96; 1-sided p-value=0.0127; 2 sided p value=0.0578).
Mean worst pain intensity was significantly lower for the EXP133 ADMIX arm compared with the BUP50 arm on Day 3 (LSM difference [SE]=−0.7 [0.34]; p=0.0215). The mean average pain intensity was significantly lower for the EXP133 ADMIX arm compared with the BUP50 arm on Days 2 and 3 (LSM difference [SE]=0.6 [0.28] and −0.5 [0.31], respectively; p values ≤0.0476).
From 0 to 24 h, 1.2% of subjects from the EXP133-ADMIX arm were opioid-free. At all other time intervals (0 to 48/72/96 hours post-surgery), no subjects were opioid-free. No subjects in the BUP50 arm were opioid free at any time interval up to 96 hours post-surgery.
Mean subject satisfaction between the EXP133-ADMIX arm (mean [SD]=8.1 [2.47]) and the BUP50 arm (mean [SD]=8.1 [2.16]) was similar (LSM difference [SE]=0.0 [0.36]; p=0.4608) at 96 hours post-surgery.
Mean AUC of worst and average pain over the last 24 hours post-surgery from POD 1 through POD 4 were significantly different (p-values ≤0.0270) between the EXP133 ADMIX arm and BUP50 arm with 6.09% and 8.97% lower values in the EXP133-ADMIX arm, respectively.
The mean current pain intensity post-surgery peaked at 24 hours post-surgery, gradually decreasing until 96 hours post-surgery in both treatment arms. From 24 hours until 54 hours post-surgery, the EXP133-ADMIX arm had a significantly lower LSM compared with the BUP50 arm at all time points (p values ≤0.0269).
In the EXP133-ADMIX arm, mean peak PK concentration was best described by an early and a late Cmax. The mean early Cmax was 467.04 ng/mL, occurring at a median time of 0.63 hours post-dose; this aligned well with the BUP50 arm, which had a Cmax of 333.89 ng/mL at a median time of 0.65 h. The mean late Cmax in the EXP133 ADMIX arm was 334.79 ng/mL, occurring at a median time of 60.94 h; by comparison, the mean bupivacaine concentration at 60 hours in the BUP50 arm was 43.25 ng/mL.
Mean extent of exposure as described by AUC0-last and AUC0-∞ were 25038.63 h×ng/mL and 25109.09 h×ng/mL in the EXP133-ADMIX arm, respectively and 7283.31 h×ng/mL and 7446.40 h×ng/mL (3510.207) in the BUP50 arm. The mean t½e1, CL/F, and Vd/F values were similar between treatment arms.
In both treatment arms, the first time point at which knee extension was not present in the majority of subjects (EXP133 ADMIX: 58.3%; BUP50: 71.4%) was 8 hours post dose.
The median duration of sensory block was about 2-fold longer in the EXP133-ADMIX arm (22.90 hours) compared with the BUP50 arm (11.63 hours). However, difference comparisons versus bupivacaine for the duration of sensory block did not achieve statistical significance (p-values ≥0.1222). Sensory block was observed in ≥50% of subjects in each treatment arm at 30 min post dose. All subjects in the BUP50 arm regained normal sensory function by 24 hours post-dose, whereas, this was not attained by all EXP133 ADMIX subjects until 144 hours post dose.

Safety
No deaths occurred during this study and EXPAREL admixed with bupivacaine HCl was well tolerated by subjects.
A similar proportion of subjects had ≥1 TEAE in each treatment arm (EXP133-ADMIX: 89.5%; BUP50: 88.8%). The most commonly reported TEAEs overall were nausea (38.6%), constipation (36.7%), muscle spasms (12.0%), and insomnia (10.8%), with all other TEAEs being reported in <10% of subjects.
Five (3.0%) subjects had ≥1 TEAE related to study drug, with 3 subjects in the EXP133 ADMIX arm and 2 subjects in the BUP50 arm. Related TEAEs included hypokalemia, hyponatraemia, confusional state, tachycardia, and muscle spasms (1 subject each), and procedural pain (2 subjects).
Overall, TEAEs were mostly mild; 102 (61.4%) subjects had a highest severity grade of mild, 43 (25.9%) subjects had moderate TEAEs, and 3 (1.8%) subjects had severe TEAEs. The proportion of subjects with moderate and severe TEAEs were similar between treatment arms. Severe TEAEs included muscle spasms (3 subjects) and pulmonary embolism (1 subject).
Four (2.4%) subjects had AESIs that included a fall, post procedural haematoma, and dizziness (2 subjects).
Six (3.6%) subjects had ≥1 SAE, with 3 subjects in each treatment arm. All SAEs were assessed as not related or unlikely related to the study drug and included a post procedural haematoma, pneumonia, acute myocardial infarction, atrial fibrillation, pulmonary embolism, and angioedema.
One subject in the BUP50 arm that discontinued from the study following SAEs of acute myocardial infarction and atrial fibrillation.
There were minimal changes in vital signs and values were similar between treatment arms. Small decreases following surgery were noted in systolic blood pressure, diastolic blood pressure, and heart rate.
From Screening to POD7, scheduled EKG assessments were normal or not clinically significant for most subjects, with only 3 subjects (2 in EXP133-ADMIX, 1 in BUP50) having clinically significant abnormal EKG findings at 1 time point each.

### Summary of Results and Conclusions

Following surgery, subjects in the EXP133 ADMIX arm had less pain severity and lower total opioid consumption.
The median time to first rescue medication was significantly longer (4.15 hours vs. 3.63 hours) for the EXP133 ADMIX arm compared with the BUP50 arm.
In the EXP133-ADMIX arm, mean peak PK concentrations occurred at median times of 0.63 hours and 60.94 h, supporting prolonged pain control in those that received the study drug. In the BUP50 arm, a single peak PK concentration occurred at a median time of 0.65 h.
Peak bupivacaine concentrations in the EXP133-ADMIX arm and BUP50 arm aligned well with the return of motor and sensory function and suggest a prolonged analgesic effect of EXPAREL admixed with bupivacaine HCl compared with bupivacaine HCl alone. All motor and sensory function returned to normal by 168 hours post dose in both treatment arms.
No deaths occurred during this study and EXPAREL admixed with bupivacaine HCl was well tolerated by subjects.
A similar proportion of subjects had ≥1 TEAE in each treatment arm (EXP133-ADMIX: 89.5%; BUP50: 88.8%). Five (3.0%) subjects had ≥1 TEAE related to study drug, with 3 subjects in the EXP133 ADMIX arm and 2 subjects in the BUP50 arm. Six (3.6%) subjects had ≥1 SAE, with 3 subjects in each treatment arm.

### Adverse Events

An AE can be defined as any untoward medical occurrence associated with the use of a drug in humans, whether or not considered drug related. An AE (also referred to as an adverse experience) could be any unfavorable and unintended sign (e.g., abnormal laboratory finding), symptom, or disease temporally associated with the use of a drug, without any judgment about causality. An AE could arise from any use of the drug (e.g., off-label use in combination with another drug) and from any route of administration, formulation, or dose, including an overdose.

An AE could be any unfavorable and unintended change in a body structure or body function. Adverse events included any clinically significant deterioration of a subject's medical status. The AE could involve any organ or system and could be represented by the new onset or deterioration of a disease, syndrome, symptom, or physical sign, as well as by findings and results of instrumental examinations and laboratory tests. Any medically relevant and untoward change after the subject signed the ICF, including frequency or pattern changes for a fluctuating condition (e.g., migraine), was considered an AE.

An AE that occurred after administration of the study treatment was considered a treatment-emergent adverse event (TEAE). A continuous AE with varying grades of severity was to be recorded as 1 AE. The highest grade of severity experienced by that subject during the course of the continuous AE was to be recorded.

In general, the severity of an AE was to be categorized using the following guidelines:
Mild: An AE that was easily tolerated by the subject, caused minimal discomfort, and did not interfere with everyday activities.
Moderate: An AE that was discomforting and interfered with normal everyday activities.
Severe: An AE that prevented normal everyday activities.

Assessment of the relationship of the AE to study drug after careful medical consideration on a case-by-case basis. General guidelines are provided below:
Unrelated: A causal relationship between the study drug and the AE could be easily ruled out (e.g., based on the temporal sequence, absence of a reasonable pathophysiological mechanism, or direct evidence of actual cause).
Unlikely: A clinical event with a temporal relationship to study drug administration that made a causal relationship improbable and in which other drugs, chemicals, or underlying disease provided a plausible explanation.
Possible: A clinical event with a reasonable time sequence to administration of the study drug but that could also be explained by a concurrent disease or other drugs or chemicals.
Probable: A clinical event with a reasonable time sequence to administration of the study drug unlikely to be attributed to a concurrent disease or other drugs or chemicals and that followed a clinically reasonable response on withdrawal (dechallenge).
Definite: The pharmacological properties of the study drug(s) or of the substance class, and the course of the AE after dechallenge and, if applicable, after rechallenge, and/or specific test indicated involvement of the study drug(s) in the occurrence/worsening of the AE, and no indication of other causes existed.

Based on review of all peripheral nerve blocks, the following conditions were considered to be Adverse Events of Special Interest (AESIs) upon review of the AEs:
Falls
Persistent tingling
Persistent numbness
Persistent weakness
Hypersensitivity
Seizures
Tremors
Dizziness
Hematoma formation
Cardiovascular depression
Dyspnea
Cardiovascular arrest
Altered sensorium
Visual disturbances
Local anesthetic systemic toxicity

Adverse events of special interest classified as persistent referred to any condition (e.g., tingling, numbness, or sensory/motor weakness that affected the nerve block region, after the study drug administration) that persisted for >168 h from the time of onset.

A Serious Adverse Event (SAE) was defined as an AE or suspected adverse reaction that, in the view of either the Investigator or Sponsor, resulted in any of the following outcomes:
Death: Any event that resulted in a subject's death was reported as an SAE. However, death, in and of itself, was not an AE; it was an outcome. The cause of death was the AE. Therefore, the Investigator was to make every effort to obtain and document the cause of death for all subjects who died during the study. If, despite all efforts, the cause of death remained unknown, the AE was to be documented as an “unspecified fatal event.”
Life-threatening: An AE was considered life-threatening if, in the view of either the Investigator or Sponsor, its occurrence placed the subject at immediate risk of death. It did not include an AE that, had it occurred in a more severe form, might have caused death.
Inpatient hospitalization or prolongation of existing hospitalization: Hospitalization, in and of itself, did not represent an SAE. It was the AE leading to the subject's hospitalization that became “serious” when it required inpatient care. Consequently, an SAE was not to be reported in case of preplanned hospitalizations for a pre-existing condition that did not worsen during the study. However, any medical condition that delayed a subject's discharge from the hospital (i.e., prolonged hospitalization) or required the subject to be readmitted was reported as an SAE.
Persistent or significant incapacity: A substantial disruption of a person's ability to conduct normal life functions.
Congenital anomaly/birth defect: If suspected that exposure to the study drug prior to conception or during pregnancy may have resulted in an adverse outcome in the child.
Medically significant: Important medical events that did not result in death, were not life-threatening, or did not require hospitalization, could be considered serious when, based upon appropriate medical judgment, they could jeopardize the subject and might have required medical or surgical intervention to prevent one of the outcomes listed in this definition.

Any SAE or death that occurred at any time after the subject signed the ICF through POD14, whether or not related to study drug, was to be reported within 24 h of discovery.

1. A method of administering regional analgesia by adductor canal block to a patient, the method comprising:
a) selecting an entry point of an injection needle in a leg of a patient by ultrasound transducer;
b) inserting the injection needle into the patient at the entry point;
c) identifying a first nerve in the adductor canal of the leg of the patient;
d) administering to the first nerve saline and a pharmaceutical composition;
e) identifying a second nerve in the adductor canal of the leg of the patient;
f) administering to the second nerve saline and the pharmaceutical composition, wherein about 133 mg of the pharmaceutical composition is administered in total;
wherein the first nerve and second nerve are selected from the group consisting of the nerve to vastus medialis (NVM) and the saphenous nerve, and wherein the first nerve is not the second nerve, and
wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome,
thereby administering regional analgesia by adductor canal block to the patient.

2. The method of claim 1, wherein the injection needle is connected to a peripheral nerve stimulator (PNS).

3. The method of claim 2, wherein the PNS is used to identify the first nerve and/or the second nerve.

4. The method of claim 1, wherein the injection needle is a 100 mm, 21-gauge needle.

5. The method of claim 1, wherein administering the pharmaceutical composition comprises administering about 10 mL of the pharmaceutical composition to each of the first nerve and the second nerve.

6. The method of claim 1, wherein the method comprises administering about 20 mL total of the pharmaceutical composition.

7. The method of claim 1, wherein the multivesicular liposomes comprise:
bupivacaine or a salt thereof;
phosphoric acid;
a lipid component comprising at least one amphipathic lipid and at least one neutral lipid lacking a hydrophilic head group; and,
a cholesterol and/or a plant sterol,
wherein said multivesicular liposomes are made by a process comprising:
a) preparing a first aqueous component comprising phosphoric acid;
b) preparing a lipid component comprising at least one organic solvent, at least one amphipathic lipid, and at least one neutral lipid lacking a hydrophilic head group;
c) mixing said first aqueous component and said lipid component to form a water-in-oil emulsion, wherein at least one component of the lipid component and the first aqueous component comprises bupivacaine or a salt thereof;
d) mixing said water-in-oil emulsion with a second aqueous component to form solvent spherules; and
e) removing the organic solvent from the solvent spherules to form multivesicular liposomes encapsulating bupivacaine phosphate.

8. The method of claim 1, further comprising administering local anesthetic to the patient in the space between the popliteal artery and the posterior capsule of the knee.

9. A method of reducing opioid consumption over a period of at least 24 hours, the method comprising:
a) selecting an entry point of an injection needle in a leg in a patient by ultrasound transducer;
b) advancing a needle tip of the injection needle within a region between a vastus medialis muscle and a sartorius muscle of the patient along a trajectory that extends between the entry point and a superficial femoral artery;
c) identifying a first nerve in the adductor canal of the leg of the patient;
d) administering to the first nerve through the injection needle saline and about 10 mL of a pharmaceutical composition;
e) identifying a second nerve in the adductor canal of the leg of the patient;
f) administering to the second nerve through the injection needle saline and about 10 mL of the pharmaceutical composition, wherein about 133 mg of the pharmaceutical composition is administered in total;
wherein the first nerve and second nerve are selected from the group consisting of the nerve to vastus medialis (NVM) and the saphenous nerve, and wherein the first nerve is not the second nerve, and
wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome,
thereby reducing opioid consumption over a period of at least 24 hours.

10. The method of claim 9, wherein the injection needle is connected to a peripheral nerve stimulator (PNS).

11. The method of claim 10, wherein the PNS is used to identify the first nerve and/or the second nerve.

12. The method of claim 9, wherein the injection needle is a 100 mm, 21-gauge needle.

13. The method of claim 9, wherein the multivesicular liposomes comprise:
bupivacaine or a salt thereof;
phosphoric acid;
a lipid component comprising at least one amphipathic lipid and at least one neutral lipid lacking a hydrophilic head group; and,
a cholesterol and/or a plant sterol,
wherein said multivesicular liposomes are made by a process comprising:
a) preparing a first aqueous component comprising phosphoric acid;
b) preparing a lipid component comprising at least one organic solvent, at least one amphipathic lipid, and at least one neutral lipid lacking a hydrophilic head group;
c) mixing said first aqueous component and said lipid component to form a water-in-oil emulsion, wherein at least one component of the lipid component and the first aqueous component comprises bupivacaine or a salt thereof;
d) mixing said water-in-oil emulsion with a second aqueous component to form solvent spherules; and
e) removing the organic solvent from the solvent spherules to form multivesicular liposomes encapsulating bupivacaine phosphate.

14. The method of claim 9, further comprising administering local anesthetic to the human patient in the space between the popliteal artery and the posterior capsule of the knee.

15. The method of claim 14, wherein the local anesthetic comprises bupivacaine HCl.

16. The method of claim 14, wherein the space between the popliteal artery and the posterior capsule of the knee is determined by ultrasound.

17. A method of administering an adductor canal block to a patient, the method comprising:
a) selecting an entry point of an injection needle in a leg of the patient;
b) inserting the injection needle into the leg of the patient at the entry point;
c) identifying a first nerve in the adductor canal of the leg of the patient;
d) administering to the first nerve a pharmaceutical composition;
e) identifying a second nerve in the adductor canal of the leg of the patient;
f) administering to the second nerve the pharmaceutical composition wherein about 133 mg of the pharmaceutical composition is administered in total;
wherein the first nerve and second nerve are selected from the group consisting of the nerve to vastus medialis (NVM) and the saphenous nerve, and wherein the first nerve is not the second nerve, and
wherein the pharmaceutical composition comprises multivesicular liposomes comprising: at least one amphipathic lipid, at least one neutral lipid, and bupivacaine phosphate, wherein the bupivacaine phosphate is encapsulated within the multivesicular liposome,
thereby administering the adductor canal block to the patient.

18. (canceled)

19. The method of claim 17, wherein the needle is connected to a peripheral nerve stimulator (PNS), which is tuned to 2 hertz and between 0.5 to 1.0 mA.

20. The method of claim 18, wherein the PNS is used to identify the first nerve and/or the second nerve.

21. The method of claim 17, wherein the injection needle is a 100 mm, 21-gauge needle.

22. The method of claim 17, wherein inserting the injection needle into the leg of the patient comprises advancing a needle tip of the injection needle within a region between a vastus medialis muscle and a sartorius muscle of the patient along a trajectory that extends between the entry point and a superficial femoral artery.

23. The method of claim 17, wherein the multivesicular liposomes comprise:
bupivacaine or a salt thereof;
phosphoric acid;
a lipid component comprising at least one amphipathic lipid and at least one neutral lipid lacking a hydrophilic head group; and,
a cholesterol and/or a plant sterol,
wherein said multivesicular liposomes are made by a process comprising:
a) preparing a first aqueous component comprising phosphoric acid;
b) preparing a lipid component comprising at least one organic solvent, at least one amphipathic lipid, and at least one neutral lipid lacking a hydrophilic head group;
c) mixing said first aqueous component and said lipid component to form a water-in-oil emulsion, wherein at least one component of the lipid component and the first aqueous component comprises bupivacaine or a salt thereof;
d) mixing said water-in-oil emulsion with a second aqueous component to form solvent spherules; and
e) removing the organic solvent from the solvent spherules to form multivesicular liposomes encapsulating bupivacaine phosphate.

24. The method of claim 17, wherein administering the pharmaceutical composition comprises administering about 10 mL of the pharmaceutical composition to each of the first nerve and the second nerve.

25. The method of claim 17, wherein the method comprises administering about 20 mL total of the pharmaceutical composition.

26. The method of claim 17, further comprising administering local anesthetic to the patient in the space between the popliteal artery and the posterior capsule of the knee.

27. A method of increasing the amount of time before opioid medication is administered to a patient, the method comprising:
a) selecting an entry point of an injection needle in a leg of a patient by ultrasound transducer, wherein the entry point comprises a region adjacent to a superficial femoral artery in the middle of a sartorius muscle;
b) advancing a needle tip of the injection needle within a region between a vastus medialis muscle and a sartorius muscle of the patient along a trajectory that extends between the entry point and a superficial femoral artery;
c) identifying a nerve to vastus medialis (NVM) in the patient;
d) administering to the NVM through the injection needle about 10 mL of a multivesicular liposome pharmaceutical composition;
e) identifying a saphenous nerve, wherein identifying a saphenous nerve in the patient comprises penetrating a vasto-adductor membrane with the needle tip and advancing the needle tip to a location anterior to the superficial femoral artery;
f) administering to the saphenous nerve through the injection needle about 10 mL of a multivesicular liposome pharmaceutical composition to the saphenous nerve, wherein about 133 mg of the pharmaceutical composition is administered in total;
wherein the multivesicular liposome pharmaceutical composition comprises:
bupivacaine or a salt thereof;
phosphoric acid;
a lipid component comprising at least one amphipathic lipid and at least one neutral lipid lacking a hydrophilic head group; and,
a cholesterol and/or a plant sterol,
thereby increasing the amount of time before opioid medication is administered to the patient.

28. (canceled)

29. The method of claim 27, wherein the needle is connected to a peripheral nerve stimulator (PNS), which is tuned to 2 hertz and between 0.5 to 1.0 mA.

30. The method of claim 27, further comprising administering local anesthetic to the patient in the space between the popliteal artery and the posterior capsule of the knee.
